(19) United States
(12) Patent Application Publication
(10) Pub. No.: US 2025/0099541 A1
(43) Pub. Date: Mar. 27, 2025

(54) COMPOSITIONS AND METHODS FOR REDUCING OXIDATION OF IGF-1/IGFBP

(71) Applicant: Oak Hill Bio Ltd, Altrincham (GB)

(72) Inventors: Nazila SALAMAT-MILLER, Lexington, MA (US); Kris LOWE, Lexington, MA (US); Michael HAHN, Lexington, MA (US); Katherine TAYLOR, Lexington, MA (US)

(21) Appl. No.: 18/730,442

(22) PCT Filed: Jan. 18, 2023
(86) PCT No.: PCT/EP2023/051110

§ 371 Date: Jul. 19, 2024

Related U.S. Application Data
(60) Provisional application No. 63/300,860, filed on Jan. 19, 2022.

(51) Int. Cl. A61K 38/18; C07K 14/475

(52) U.S. Cl. CPC A61K 38/18; C07K 14/475

(57) ABSTRACT

The present invention provides, among other things, compositions and methods for reducing oxidation of a complex comprising insulin-like growth factor-1 (IGF-1) and insulin-like growth factor binding protein-3 (IGFBP-3), for treating diseases and complications of prematurity. In some embodiments, the methods comprise adding antioxidants. In some embodiments, the antioxidants are methionine or sodium thiosulfate. In some aspects, the compositions provided are stable, pure and potent compositions comprising IGF-1 and IGFBP-3. In some aspects, the formulation is lyophilized.

The present disclosure relates to a stable formulation of Mecasermin rinfabate (rhIGF-1/rhIGFBP-3), wherein oxidation of one or more species is minimized on storage, for example 2-8° C. and/or 25° C., methods of manufacturing said formulation, and use of same in treatment, in particular treatment of infants, such as premature infants.

### BACKGROUND

Mecasermin rinfabate is a complex of recombinant human insulin-like growth factor-1 (rhIGF-1) and recombinant human insulin-like growth factor binding protein-3 (rhIGFBP-3) used to treat diseases and complications of prematurity, including intraventricular hemorrhage, bronchopulmonary dysplasia and/or chronic lung disease of prematurity.

There is a need for an formulation optimized specifically for greater stability of the therapeutic protein product at 2-8° C., 25° C. or 40° C. for example, during storage for about 3-6 months or longer.

Very dilute formulations are required to treat infants and especially premature babies. Mecasermin rinfabate is typically administered to preterm infants through continuous intravenous infusion. Surprisingly the present inventors have established that in dilute formulations of the concentrations the complex oxidizes more rapidly.

### SUMMARY

The present invention provides in part, compositions and methods for reducing oxidation of a formulation comprising a protein complex comprising insulin-like growth factor-1 (IGF-1) and insulin-like growth factor binding protein-3 (IGFBP-3). Without wishing to be bound by any particular theory, it is contemplated that methods to reduce oxidized species (e.g. addition of antioxidants, lyophilization, and/or a single-use bag) provide an improved formulation with greater potency and increased stability.

The present disclosure is summarized in the following numbered paragraphs:
1. A composition comprising an isolated protein complex comprising insulin-like growth factor 1 (IGF-1) and insulin-like growth factor binding protein (such as IGFBP-3), for example at a molar ratio of 1:1, wherein the composition comprises an antioxidant, and wherein the oxidized IGF-1 species does not increase more than 50% upon storage at a temperature of about 25° C. after three months.
2. The composition of paragraph 1, wherein the oxidized IGF-1 species does not increase more than 25% upon storage at a temperature of about 25° C. after three months.
3. The composition of paragraph 1 or 2, wherein the oxidized IGF-1 species does not increase more than 10% upon storage at a temperature of about 25° C. after three months.
4. The composition of paragraph 1, wherein the oxidized IGF-1 species does not substantially increase upon storage at a temperature of about 25° C. after three months.
5. A composition comprising an isolated protein complex comprising insulin-like growth factor 1 (IGF-1) and insulin-like growth factor binding protein (such as IGFBP-3), for example at a molar ratio of 1:1, wherein the composition comprises an antioxidant, and wherein the % of the oxidized IGF-1 species does not increase more than 20% upon storage at a temperature of between 2-8° C. after three months.
6. The composition of paragraph 5, wherein the oxidized IGF-1 species does not increase more than 10% upon storage at a temperature of between 2-8° C. after three months.
7. The composition of paragraph 6, wherein the oxidized IGF-1 species does not substantially increase upon storage at a temperature of between 2-8° C. after three months.
8. The composition of any one of preceding paragraphs, wherein the antioxidant is methionine.
9. The composition of any one of preceding paragraphs, wherein the composition comprises methionine at a concentration of between 0.2 mM and 50 mM.
10. The composition of paragraph 9, wherein the composition comprises methionine at a concentration of 0.5 mM.
11. The composition of paragraph 9, wherein the composition comprises methionine at a concentration of 5 mM.
12. The composition of any one of paragraphs 1-7, wherein the antioxidant is sodium thiosulfate.
13. The composition of any one of preceding paragraphs, wherein the composition comprises the sodium thiosulfate at a concentration of between 0.2 mM and 50 mM.
14. The composition of paragraph 13, wherein the composition comprises the sodium thiosulfate at a concentration of 0.5 mM.
15. The composition of paragraph 13, wherein the composition comprises the sodium thiosulfate at a concentration of 5 mM.
16. The composition of any one of the preceding paragraphs, wherein less than 1% of the IGF-1 exists as oxidized species in the protein complex upon storage for 3 months.
17. The composition of any one of the preceding paragraphs, wherein less than 0.6% of the IGF-1 exists as oxidized species in the protein complex upon storage for 3 months.
18. The composition of any one of the preceding paragraphs, wherein the amount of oxidized species is determined by RP-UPLC or SEC-HPLC.
19. The composition of any one of preceding paragraphs, wherein the IGFBP-3 comprises less than 5% of trisulfide variants.
20. The composition of paragraph 19, wherein the IGFBP-3 comprises less than 2% of trisulfide variants.
21. The composition of paragraph 19, wherein the IGFBP-3 comprises less than 1% of trisulfide variants.
22. The composition of paragraph 19, wherein the IGFBP-3 comprises less than 0.5% of trisulfide variants.
23. A method for manufacturing an insulin-like growth factor 1 (IGF-1) protein complex comprising steps of: providing a composition comprising recombinant IGF-1; adding an antioxidant to the composition and purifying the IGF-1 from the composition.
24. The method of paragraph 23, wherein the antioxidant is methionine.
25. The method of paragraph 23, wherein the methionine is added at a concentration of between 0.2 mM and 10 mM.
26. The method of paragraph 23, wherein the methionine is added at a concentration of 0.5 mM.
27. The method of paragraph 23, wherein the methionine is added at a concentration of 5 mM.
28. The method of paragraph 23, wherein the antioxidant is sodium thiosulfate.
29. The method of paragraph 28, wherein the sodium thiosulfate is added at a concentration of between 0.2 mM and 10 mM.
30. The method of paragraph 28, wherein the sodium thiosulfate is added at a concentration of 0.5 mM.
31. The method of paragraph 28, wherein the sodium thiosulfate is added at a concentration of 5 mM.
32. The method of any one of paragraphs 23 to 31, wherein the oxidized IGF-1 species in the purified IGF-1 composition does not increase more than 50% upon storage at a temperature of about 25° C. after three months.
33. The method of any one of paragraphs 23 to 31, wherein the oxidized IGF-1 species in the purified IGF-1 composition does not increase more than 20% upon storage at a temperature of about 25° C. after three months.
34. The method of any one of paragraphs 23 to 31, wherein the oxidized IGF-1 species in the purified IGF-1 composition does not increase more than 10% upon storage at a temperature of about 25° C. after three months.
35. The method of any one of paragraphs 23 to 31, wherein the oxidized IGF-1 species in the purified IGF-1 composition does not increase substantially upon storage at a temperature of about 25° C. after three months.
36. The method of any one of paragraphs 23 to 31, wherein the oxidized IGF-1 species in the purified IGF-1 composition does not increase more than 20% upon storage at a temperature of between 2-8° C. after three months.
37. The method of any one of paragraphs 23 to 31, wherein the amount of oxidized IGF-1 species in the purified IGF-1 composition does not increase more than 10% upon storage at a temperature of between 2-8° C. after three months.
38. The method of any one of paragraphs 23 to 31, wherein the amount of oxidized IGF-1 species in the purified IGF-1 composition does not increase substantially upon storage at a temperature of between 2-8° C. after three months.
39. The method of any one of paragraphs 23 to 31, wherein the purified IGF-1 protein has less than 0.6% of oxidized IGF-1 species.
40. The method of any one of paragraphs 23 to 39, wherein the recombinant IGF-1 protein is expressed in E. coli.
41. The method of any one of paragraphs 23 to 40, wherein the recombinant IGF-1 protein is purified from inclusion body.
42. The method of any one of paragraphs 23 to 41, wherein the method further comprises adding insulin-like growth factor binding protein 3 (IGFBP-3) to the IGF-1 protein to form a protein complex.
43. The method of paragraph 42, wherein the protein complex comprises IGF-1 and IGFBP-3 at a molar ratio of 1:1.
44. A composition comprising an isolated protein complex comprising insulin-like growth factor 1 (IGF-1) and insulin-like growth factor binding protein 3 (IGFBP-3) manufactured by the method of any one of paragraphs 23 to 43.
45. A composition comprising an isolated protein complex comprising insulin-like growth factor 1 (IGF-1) and insulin-like growth factor binding protein 3 (IGFBP-3), for example at a molar ratio of 1:1, wherein the composition comprises a bulking agent, buffer and surfactant.
46. The composition of paragraph 45, wherein the bulking agent is sucrose or trehalose.
47. The composition of paragraph 46, wherein the bulking agent is trehalose.
48. The composition of paragraph 45, wherein the buffer is histidine or phosphate.
49. The composition of paragraph 48, wherein the buffer is between pH 5.5-6.5.

Improved stability is achieved during storage at about 25° C. (e.g. about 23° C. to about 27° C.), or storage at about 40° C. (e.g. about 38° C. to about 42° C.) for example, during storage for about 3-6 months.

In one embodiment, provided herein is an improved IGF-1/IGFBP-3 formulation comprising antioxidants (e.g. methionine and/or sodium thiosulfate).

In other embodiments, oxidized species in the formulation was reduced by lyophilization.

In other embodiments, concentrated protein is formulated in the vial, for dilution before use.

In one embodiment the disclosure provides a lyophilized formulation comprising an antioxidant (e.g. methionine and/or sodium thiosulfate).

In one embodiment the disclosure provides a high concentration formulation comprising an antioxidant (e.g. methionine and/or sodium thiosulfate).

In other embodiments, reduced accumulation of oxidized species and improved stability achieved through the use of a contact surface other than stainless steel, such as a single-use bag during compounding the complex with a formulation solution; including a formulation comprising an antioxidant (e.g. methionine and/or sodium thiosulfate) and/or a high concentration formulation.

In summary, the present invention provides, among other things, an improved formulation of IGF-1/IGFBP-3 with advantages of improved stability during storage, providing a safe and efficacious product for administration to neonates in treating diseases of prematurity.

In one aspect, provided herein is a composition comprising an isolated protein complex comprising insulin-like growth factor 1 (IGF-1) and insulin-like growth factor binding protein 3 (IGFBP-3), for example at a molar ratio of 1:1, wherein the composition comprises an antioxidant, and wherein the amount of oxidized IGF-1 species does not increase more than 50% upon storage at a temperature of about 25° C. after three months, for example does not increase more than 45%; such as does not increase more than 40%, in particular does not increase more than 35%; more specifically, does not increase more than 30%. In some embodiments, upon storage at a temperature of about 25° C. after three months, the amount of oxidized IGF-1 species does not increase more than: 25%; 20%; 15%; 10%; 5%; 4%; 3%; 2%; or 1%*.

*Each of these values represents an individual disclosure as set out in the priority filing and may be used in isolation as basis for amendment to the claims.

In some embodiments, the amount of oxidized IGF-1 species does not increase substantially upon storage at a temperature of about 25° C. after three months.

Molar ratios of IGF-1: IGFBP-3 that may be employed in formulations of the present disclosure include 1.5-0.5 IGF-1:1 IGFBP-3.

In one aspect, provided herein is a composition comprising an isolated protein complex comprising insulin-like growth factor 1 (IGF-1) and insulin-like growth factor binding protein 3 (IGFBP-3) at a molar ratio of 1:1, wherein the amount of the oxidized IGF-1 species does not increase more than 20% upon storage at a temperature of between 2-8° C. after three months.

In some embodiments, the amount of oxidized IGF-1 species upon storage at a temperature of between 2-8° C. after three months does not increase more than 15%, for example does not increase more than 10%, such as does not increase more than 5%, in particular does not increase more than 4%, more specifically does not increase more than 3%. In some embodiments, the amount of oxidized IGF-1 species does not increase more than 2% upon storage at a temperature of between 2-8° C. after three months. In some embodiments, the amount of oxidized IGF-1 species does not increase more than 1% upon storage at a temperature of between 2-8° C. after three months. In some embodiments, the amount of oxidized IGF-1 species does not increase substantially upon storage at a temperature of between 2-8° C. after three months.

In one embodiment, provided herein is a composition or method according to the present disclosure, wherein the antioxidant is methionine.

In one embodiment, provided herein is a composition or method according to the present disclosure comprising methionine (including adding antioxidant such as methionine) at a concentration of in the range (for example between) 0.2 mM and 15 mM, such as 0.2 mM and 10 mM. In one embodiment, provided herein is a composition comprising methionine at a concentration of 0.2 mM; 0.3 mM; 0.4 mM;

0.5 mM; 0.6 mM; 0.7 mM; 0.8 mM; 0.9 mM; 1 mM; 2 mM; 3 mM; 4 Mm; 5 mM; 6 mM; 7 mM; 8 mM; or 9 mM*. In one embodiment, provided herein is a composition or method according to the present disclosure comprising methionine (including adding antioxidant such as methionine) at a concentration of 10 mM.

In one embodiment, provided herein is a composition or method according to the present disclosure, wherein the antioxidant is sodium thiosulfate.

In one embodiment, provided herein is a composition or method according to the present disclosure, wherein the composition comprises sodium thiosulfate (including adding same) at a concentration of in the range (for example between) 0.2 mM and 15 mM, such as 0.2 mM and 10 mM.

In one embodiment, provided herein is a composition, wherein the composition or method according to the present disclosure comprises sodium thiosulfate at a concentration of 0.2 mM; 0.3 mM; 0.4 mM; 0.5 mM; 0.6 mM; 0.7 mM; 0.8 mM; 0.9 mM; 1 mM; 2 mM; 3 mM; 4 mM; 5 mM; 6 mM; 7 mM; 8 mM; or 9 mM*.

In one embodiment, provided herein is a composition, wherein the composition comprises sodium thiosulfate at a concentration of 10 mM.

In one embodiment, provided herein is a composition, wherein less than 5% of the IGF-1 exists as oxidized species in the protein complex upon storage for 3 months, for example wherein less than 4% such as, wherein less than 3% in particular, wherein less than 2%, in particular wherein less than 1%.

In one embodiment, provided herein is a composition, wherein less than 0.9% of the IGF-1 exists as oxidized species in the protein complex upon storage for 3 months, for example less than 0.8% of the IGF-1 exists as oxidized species, such as less than 0.7% of the IGF-1 exists as oxidized species, in particular less than 0.6% of the IGF-1 exists as oxidized species in the protein complex upon storage for 3 months, more specifically less than 0.5% of the IGF-1 exists as oxidized species. In one embodiment, provided herein is a composition, wherein less than 0.4% of the IGF-1 exists as oxidized species in the protein complex upon storage for 3 months. In one embodiment, provided herein is a composition, wherein less than 0.3% of the IGF-1 exists as oxidized species in the protein complex upon storage for 3 months. In one embodiment, provided herein is a composition, wherein less than 0.2% of the IGF-1 exists as oxidized species in the protein complex upon storage for 3 months. In one embodiment, provided herein is a composition, wherein less than 0.1% of the IGF-1 exists as oxidized species in the protein complex upon storage for 3 months.

In one embodiment, provided herein is a composition wherein the amount of oxidized species is determined by Reversed Phase-Ultra Performance Liquid Chromatography (RP-UPLC).

In one embodiment, provided herein is a composition wherein the amount of oxidized species is determined by Size Exclusion Chromatography-HPLC separation modes (SEC-HPLC).

In one embodiment, provided herein is a composition wherein the IGFBP-3 comprises less than 5% of trisulfide variants, for example less than 4% of trisulfide variants, such as less than 3% of trisulfide variants, in particular less than 2% of trisulfide variants, more specifically less than 1% of trisulfide variants.

In one aspect, provided herein is a method for manufacturing an insulin-like growth factor 1 (IGF-1) protein complex comprising steps of providing a composition comprising recombinant IGF-1, adding an antioxidant to the composition, and purifying the IGF-1 from the composition.

In one embodiment, provided herein is a method comprising adding an antioxidant, wherein the antioxidant is methionine.

In one embodiment, provided herein is a method comprising adding an antioxidant, wherein the antioxidant is sodium thiosulfate.

In one embodiment, provided herein is a method, wherein the amount of oxidized IGF-1 species in the purified IGF-1 composition does not increase more than 50% upon storage at a temperature of about 25° C. after three months, for example does not increase more than 45%, such as does not increase more than 40%, in particular does not increase more than 35%, more specifically does not increase more than 30%.

In one embodiment, provided herein is a method, wherein the amount of oxidized IGF-1 species in the purified IGF-1 composition does not increase more than 25%; 20%; 15%; 10%; 5%; 3%; 2%* upon storage at a temperature of about 25° C. after three months. In one embodiment, provided herein is a method, wherein the amount of oxidized IGF-1 species in the purified IGF-1 composition does not increase more than 1% upon storage at a temperature of about 25° C. after three months. In one embodiment, provided herein is a method, wherein the amount of oxidized IGF-1 species in the purified IGF-1 composition does not increase substantially upon storage at a temperature of about 25° C. after three months.

In some embodiments, provided herein is method, wherein the amount of oxidized IGF-1 species in the purified IGF-1 composition does not increase more than 20%; 15%; 10%; 5%; 4%; 3%; 2%* upon storage at a temperature of between 2-8° C. after three months. In some embodiments, the amount of oxidized IGF-1 species does not increase more than 1% upon storage at a temperature of between 2-8° C. after three months. In some embodiments, the amount of oxidized IGF-1 species does not increase substantially upon storage at a temperature of between 2-8° C. after three months.

In some embodiments, provided herein is a method, wherein the purified IGF-1 protein has less than 5%; 4%; 3%; 2%; 1%; 0.9%; 0.8%; 0.7%; 0.6%; 0.5%; 0.4%; 0.3%; 0.2%* of oxidized IGF-1 species.

In some embodiments, provided herein is a method, wherein the purified IGF-1 protein has less than 0.1% of oxidized IGF-1 species.

In some embodiments, provided herein is a method, wherein the recombinant IGF-1 protein is expressed in E. coli. In some embodiments, provided herein is a method, wherein the recombinant IGF-1 protein is purified from an inclusion body.

In some embodiments, the method further comprises adding insulin-like growth factor binding protein (such as IGFBP-3) to the IGF-1 protein to form a protein complex. In some embodiments, the protein complex comprises IGF-1 and IGFBP-3 in a range 0.75 to 1.25:1 or 1:0.75-1.25 such as in equimolar amounts. In some embodiments, the protein complex comprises IGF-1 and IGFBP-3 in 0.75 to 1.25 molar ratio. In some embodiments, the protein complex comprises IGF-1 and IGFBP-3 in 1:1 molar ratio. In some embodiments, the protein complex comprises IGF-1 and IGFBP-3 in 1.25-0.75 molar ratio.

In some embodiments, the composition comprises an isolated protein complex comprising insulin-like growth factor 1 (IGF-1) and insulin-like growth factor binding protein 3 (IGFBP-3) manufactured by the method.

In one embodiment the compositions of the present disclosure comprise a surfactant, for example selected from polysorbate (such as polysorbate 80 or 20) or Triton-X, in particular 0.01% or less, such as 0.008%, 0.0075%, 0.007%, 0.006%, 0.005%, 0.004%, 0.003% or 0.

In one embodiment % of the formulation/composition is w/w or w/v or v/v.

In some aspects, provided herein is a composition according to any aspect of the disclosure comprising a protein complex comprising recombinant insulin-like growth factor 1 (rIGF-1) and recombinant insulin-like growth factor binding protein 3 (rIGFBP-3) in equimolar amounts, polysorbate surfactant such as 80 or 20, in particular polysorbate 20 at a concentration in the range 0.0025 to 0.0075 such as a concentration of about 0.005%, and a buffer comprising sodium acetate, acetic acid, and/or sodium chloride, wherein the composition has a pH of about 5.3-5.8, wherein the rIGF-1/IGFBP-3 is at a concentration of 45 to 55 micrograms/mL, about 50 micrograms/mL, and wherein less than 0.6% of the IGF-1 exists as oxidized species, for example wherein the composition comprises an antioxidant, such as methionine and/or sodium thiosulfate, in particular as described elsewhere herein.

In one embodiment there is provided a lyophilized form of a composition according to any disclosure/embodiment described herein, in particular comprising a bulking agent.

In one aspect, provided herein is a lyophilized formulation comprising an isolated protein complex comprising insulin-like growth factor 1 (IGF-1) and insulin-like growth factor binding protein (such as IGFBP-3) at a molar ratio of 1:1, a bulking agent, buffer and surfactant.

In one embodiment, the bulking agent is sucrose or trehalose. In one embodiment, the bulking agent is sucrose. In one agent, the bulking agent is trehalose.

In one embodiment, the buffer is histidine or phosphate. In one embodiment, the buffer is histidine. In one embodiment, the buffer is phosphate. In one embodiment, the buffer is between pH 5.5-6.5.

In one embodiment a formulation according to the present disclosure comprises one or more amino acids, for example independently selected from an essential amino acid.

In one embodiment there is provide use of a composition according to the disclosure in treatment.

In one embodiment there is provided use of a composition according to the present disclosure in the manufacture of a medicament for treatment, in particular for the treatment of an infant, such as premature infant, a low gestation weight infant and/or an infant following pre-eclampsia, in particular for the treatment or prevention on BPD, ROP and/or IVH.

It is to be understood that while the invention has been described in conjunction with the preferred specific embodiments thereof that the foregoing description as well as the examples that follow are intended to illustrate and not limit the scope of the invention. Other aspects, advantages and modifications within the scope of the invention will be apparent to those skilled in the art to which the invention pertains.

In one embodiment the composition of the present disclosure is liquid.

In one embodiment the composition of the present disclosure is stored at 2-8° C.

One embodiment comprises reconstitution of a lyophilized formulation according to the present disclosure, for example with an aqueous solution (including water for injection, saline or glucose, an isotonic solution).

In one embodiment a formulation according to the present disclosure is isotonic before administration to a patient, for example because it was reconstituted with an aqueous solution (such as an isotonic solution) or because the final drug product is isotonic.

In one embodiment a composition according to the present disclosure does not comprise benzalkonium chloride. This excipient is contraindicated for premature infants. In one embodiment formulations of the present disclosure do not comprise a preservative.

In one embodiment the formulation/composition according to the present disclosure does not comprise any ingredients that are toxic to an infant, for example a premature infant.

In one embodiment stability refers to chemical stability, i.e. stabilization of chemical degradation.

In one embodiment stability refers to physical stability, for example often physical stability results in aggregation, adsorption and/or changes in solubility (particularly increased insolubility).

In one embodiment the compositions of the present disclosure minimize adsorption and/or loss of protein (i.e. IGF-1 and/or IGFBP, such as IGFBP-3) during manufacturing and/or administration.

In embodiment stability refers to thermal stability, for example the ability to withstand increased and/or reduced temperature, such as the formulations of the present disclosure are able withstand room temperature (such as about 25 to 27° C.) for at least 24 hours, in particular provide adequate stability for administration, especially continuous administration.

In one embodiment the compositions according to the disclosure are resistant to decomposition or change caused by humidity.

In one embodiment a composition according to the present disclosure is provided as a unit dose, for example a dose for a 24 hour period, in particular a dose for a premature infant.

In one embodiment there is provided a composition of the present disclosure in a single-usebag, for example a liquid formulation, in particular wherein the formulation is suitable/ready for administration. This is advantageous because it minimizes contact of the formulation with surfaces that can catalyze oxidation and also minimized contamination, infect to the patient.

In one embodiment there is provided a concentrated liquid formulation according to the present disclosure is provided in a vial, in particular a glass vial.

In one embodiment there is provided a lyophilized formulation according to the present disclosure in a vial, for example a glass vial.

In one embodiment the content of vial or container, (including a bag) filled with a formulation according to the present disclosure, is under an inert gas, such as nitrogen.

In one embodiment deamination degradations pathways are minimized in the protein complex formulations according to the present disclosure.

In one embodiment formulations of the present disclosure have a shelf-life of at least 3 months, 6 months, 9 months, 12 months, 18 months or 24 months, when stored under appropriate conditions.

In one embodiment the formulations of the present disclosure are sterilized by filtration, for example where the loss of protein complex is minimized during filtration by the formulation of the present disclosure.

In one embodiment drug product has an outer foil packaging component to minimize moisture ingression and/or exposure to light.

In one embodiment protein complexes formulated (such as a liquid formulation) and/or manufactured according to the present disclosure perform well in stress testing, for example physical stress testing such as agitation testing, shear testing, flocculation analysis, liquid air interface analysis, dissolution testing or similar.

In one embodiment there is provided a formulation and/or drug product obtainable from a method disclosed herein.

### DETAILED DESCRIPTION

The present invention provides, among other things, methods and compositions for reducing oxidation of a formulation comprising a protein complex comprising insulin-like growth factor-1 (IGF-1) and insulin-like growth factor binding protein (such as IGFBP-3). Methods to reduce oxidized species (e.g. addition of antioxidants, lyophilization, a single-use bag) provide an improved formulation with greater potency and increased stability. Improved stability is achieved during storage at about 25° C. (e.g. about 23° C. to about 27° C.), or storage at about 40° C. (e.g. about 38° C. to about 42° C.) for example, during storage for about 3-6 months. In some embodiments, the present invention provides an improved IGF-1/IGFBP-3 formulation comprising antioxidants (e.g. methionine or sodium thiosulfate). In other embodiments, the present invention provides lyophilized formulations with reduced oxidized species.

In other embodiments, a high concentration of protein was formulated in the vial, for dilution before use.

In other embodiments, reduced accumulation of oxidized species and improved stability achieved through the use of a contact surface other than stainless steel, such as a single-use bag during compounding the complex with a formulation solution. In summary, the present invention provides, among other things, improved formulations of IGF-1/IGFBP-3 with advantages of improved stability during storage, providing a safe and efficacious product for administration to neonates in treating diseases of prematurity.

In some aspects, provided herein is a composition comprising an isolated protein complex comprising insulin-like growth factor 1 (IGF-1) and insulin-like growth factor binding protein 3 (IGFBP-3) at a molar ratio of 1:1, wherein the composition comprises an antioxidant, and wherein the amount of oxidized IGF-1 species does not increase more than 50% upon storage at a temperature of about 25° C. after three months.

In one aspect, provided herein is a composition comprising an isolated protein complex comprising insulin-like growth factor 1 (IGF-1) and insulin-like growth factor binding protein 3 (IGFBP-3) at a molar ratio of 1:1, wherein the amount of the oxidized IGF-1 species does not increase more than 20% upon storage at a temperature of between 2-8° C. after three months.

The present invention, provides in some embodiments, a composition wherein the antioxidant is methionine, comprising methionine, for example, at a concentration of between 0.2 mM and 10 mM. In some embodiments, provided herein is a composition, wherein the antioxidant is sodium thiosulfate, for example, at a concentration of between 0.2 mM and 10 mM. In some embodiments, provided herein is a composition, wherein less than 5% of the IGF-1 exists as oxidized species in the protein complex upon storage for 3 months. The amount of oxidized species is determined by Reversed Phase-Ultra Performance Liquid Chromatography (RP-UPLC) or by Size Exclusion Chromatography-HPLC separation modes (SEC-HPLC). In some embodiments, provided herein is a composition wherein the IGFBP-3 comprises less than 5% of trisulfide variants.

In one aspect, provided herein is a method for manufacturing an insulin-like growth factor 1 (IGF-1) protein complex comprising steps of providing a composition comprising recombinant IGF-1, adding an antioxidant to the composition, and purifying the IGF-1 from the composition.

Provided herein is a method of manufacturing an IGF-1/IGFBP-3 complex comprising adding an antioxidant, wherein the antioxidant is methionine, wherein the antioxidant is methionine added at a concentration of between 0.2 mM and 10 mM. In one embodiment, provided herein is a method comprising adding an antioxidant, wherein the antioxidant is sodium thiosulfate, wherein the antioxidant is sodium thiosulfate added at a concentration of between 0.2 mM and 10 mM.

The present invention, in some embodiments, provides a method wherein the amount of oxidized IGF-1 species in the purified IGF-1 composition does not increase more than 50% upon storage at a temperature of about 25° C. after three months. In some embodiments, provided herein is method, wherein the amount of oxidized IGF-1 species in the purified IGF-1 composition does not increase more than 20% upon storage at a temperature of between 2-8° C. after three months. In some embodiments, provided herein is a method, wherein the purified IGF-1 protein has less than 5% of oxidized IGF-1 species. In some embodiments, provided herein is a method, wherein the recombinant IGF-1 protein is expressed in E. coli. In some embodiments, provided herein is a method, wherein the recombinant IGF-1 protein is purified from an inclusion body.

In some embodiments, the method further comprises adding insulin-like growth factor binding protein 3 (IGFBP-3) to the IGF-1 protein to form a protein complex. In some embodiments, the protein complex comprises IGF-1 and IGFBP-3 in a range 0.75 to 1.25:1 or 1:0.75-1.25 such as in equimolar amounts. In some embodiments, the protein complex comprises IGF-1 and IGFBP-3 in 0.75 to 1.25 molar ratio. In some embodiments, the protein complex comprises IGF-1 and IGFBP-3 in 1:1 molar ratio. In some embodiments, the protein complex comprises IGF-1 and IGFBP-3 in 1.25-0.75 molar ratio. In one aspect, provided herein is a method for manufacturing an insulin-like growth factor 1 (IGF-1)/IGFBP-3 protein complex comprising steps of providing a composition comprising recombinant IGF-1, recombinant IGFBP-3, adding an antioxidant to the composition, and purifying the IGF-1/IGFBP-3.

In some embodiments, the composition comprises an isolated protein complex comprising insulin-like growth factor 1 (IGF-1) and insulin-like growth factor binding protein 3 (IGFBP-3) manufactured by the method. In some aspects, provided herein is a composition comprising a protein complex comprising recombinant insulin-like growth factor 1 (rIGF-1) and recombinant insulin-like growth factor binding protein 3 (rIGFBP-3) in equimolar amounts, polysorbate 20 surfactant at a concentration of about 0.005%, and a buffer comprising sodium acetate, acetic acid, and/or sodium chloride, wherein the composition has a pH of about 5.3-5.8, wherein the rIGF-1/IGFBP-3 is at a concentration of about 50 micrograms/mL, and wherein less than 0.6% of the IGF-1 exists as oxidized species.

In one aspect, provided herein is a lyophilized formulation comprising an isolated protein complex comprising insulin-like growth factor 1 (IGF-1) and insulin-like growth factor binding protein 3 (IGFBP-3) at a molar ratio of 1:1, a bulking agent, buffer and surfactant.

In one embodiment, the bulking agent is sucrose or trehalose. In one embodiment, the bulking agent is sucrose. In one agent, the bulking agent is trehalose.

In one embodiment, the buffer is histidine or phosphate. In one embodiment, the buffer is histidine. In one embodiment, the buffer is phosphate. In one embodiment, the buffer is between pH 5.5-6.5. Various aspects of the invention are described in detail in the following sections. The use of sections is not meant to limit the invention. Each section can apply to any aspect of the invention. In this application, the use of “or” means “and/or” unless stated otherwise.

### Definitions

“Preterm” or “preterm birth” or “prematurity” or “premature infant” or “premature baby”, or grammatical equivalents, refers to birth of an infant prior to 37 weeks of gestation or weighing 10% less than the average for the infant's gestational age. For example, infants born between 22-37 weeks would be considered preterm.

In one embodiment preterm infant (or neonate) as employed herein refers to an infant born before for example with a gestational age of 37 weeks or less, such as 22 to 37 weeks (gestational age) alternatively 23 to 34 weeks or 24 to 29 weeks, in particular 23, 24, 25, 26, 27, 28, 29, 30, 31, 33, 34, 35 or 36 weeks, Generally, the patient, for example an infant, such as a preterm infant is human.

The lowest weight (not necessarily the most premature) infants may benefit the most from the therapy of the present disclosure.

Low gestations weight refers to infants in the lowest quartile of weight for the gestational age.

Infants born after preeclampsia is self-evident from the words.

“GA” or “Gestational age” is a common term to describe how far along a pregnancy has progressed, measured in weeks from the first day of the woman's last menstrual cycle to the current date.

In some embodiments, a premature infant refers to an infant that was prematurely born by at least 1 week, 2 weeks, 3 weeks, 4 weeks, 1 month, 2 months, or 3 months. In some embodiments, a premature infant refers to an infant born at less than 32 weeks of gestational age (GA). In some embodiments, a premature infant refers to an infant born at less than 28 weeks of gestational age (GA). “PMA” or “postmenstrual age” refers to gestational age plus chronological age. “CA” or “corrected age” is the chronological age reduced by the number of weeks born before 40 weeks of gestation.

“IGF-I” refers to insulin-like growth factor I from any species, including bovine, ovine, porcine, equine, and human, preferably human, and, if referring to exogenous administration, from any source, whether natural, synthetic, or recombinant, provided that it will bind IGF binding protein at the appropriate site. IGF-I can be produced recombinantly, for example, as described in WO95/04076.

An “IGFBP” or an “IGF binding protein” refers to a protein or polypeptide from the insulin-like growth factor binding protein family and normally associated with or bound or complexed to IGF-I whether or not it is circulatory (i.e., in serum or tissue). Such binding proteins do not include receptors. This definition includes IGFBP-1, IGFBP-2, IGFBP-3, IGFBP-4, IGFBP-5, IGFBP-6, Mac 25 (IGFBP-7), and prostacyclin-stimulating factor (PSF) or endothelial cell-specific molecule (ESM-1), as well as other proteins with high homology to IGFBPs. Mac 25 is described, for example, in Swisshelm et al., Proc. Natl. Acad. Sci. USA, 92:4472-4476 (1995) and Oh et al., J. Biol. Chem., 271:30322-30325 (1996). PSF is described in Yamauchi et al., Biochemical Journal, 303:591-598 (1994). ESM-1 is described in Lassalle et al., J. Biol. Chem., 271:20458-20464 (1996). For other identified IGFBPs, see, e.g., EP 375,438 published Jun. 27, 1990; EP 369,943 published May 23, 1990; WO 89/09268 published Oct. 5, 1989; Wood et al., Molecular Endocrinology, 2:1176-1185 (1988); Brinkman et al., The EMBO J., 7:2417-2423 (1988); Lee et al., Mol. Endocrinol., 2:404-411 (1988); Brewer et al., BBRC, 152:1289-1297 (1988); EP 294,021 published Dec. 7, 1988; Baxter et al., BBRC, 147:408-415 (1987); Leung et al., Nature, 330:537-543 (1987); Martin et al., J. Biol. Chem., 261:8754-8760 (1986); Baxter et al., Comp. Biochem. Physiol., 91B: 229-235 (1988); WO 89/08667 published Sep. 21, 1989; WO 89/09792 published Oct. 19, 1989; and Binkert et al., EMBO J., 8:2497-2502 (1989).

“IGFBP-3” refers to insulin-like growth factor binding protein 3. IGFBP-3 is a member of the insulin-like growth factor binding protein family. IGFBP-3 may be from any species, including bovine, ovine, porcine and human, in native-sequence or variant form, including but not limited to naturally-occurring allelic variants. IGFBP-3 may be from any source, whether natural, synthetic or recombinant, provided that it will bind IGF-I at the appropriate sites. IGFBP-3 can be produced recombinantly, as described in PCT publication WO95/04076.

Formulations for IGF-1 and an IGF binding protein (such as IGFBP-3) are disclosed in WO2022/086953, incorporated herein by reference.

A “therapeutic composition,” as used herein, is defined as comprising IGF-I, an analog thereof, or IGF-I in combination with its binding protein, IGFBP-3 (IGF-I/IGFBP-3 complex). The therapeutic composition may also contain other substances such as water, minerals, carriers such as proteins, and other excipients known to one skilled in the art. In some embodiments, the therapeutic composition comprises a surfactant such as polysorbate 20 (P20) or polysorbate 80 (P80).

“Analogs” of IGF-I are compounds having the same therapeutic effect as IGF-I in humans or animals. These can be naturally occurring analogs of IGF-I (e.g., truncated IGF-I) or any of the known synthetic analogs of IGF-I. See, for example, U.S. Pat. No. 5,473,054 for analog compounds of IGF-I.

“Antioxidants” are compounds included in the formulation that prevent or reduce the formation of oxidized species. In some embodiments, the antioxidant is selected from a reducing agent, free radical scavenger, chelating agent, chain terminator, nitrogen, etc. In some embodiments, the antioxidant is a reducing agent selected from a group consisting of L-methionine, sodium metabisulfite, sodium thiosulfate and glutathione. In some embodiments, the antioxidant is a free radical scavenger selected from a group consisting of L-histidine, L-ascorbic acid, catalase and platinum. In some embodiments, the antioxidant is a chelating agent selected from a group consisting of sodium salt of EDTA dihydrate, diethylenetriaminepentaacetic acid (DTPA) and citric acid. In some embodiments, the antioxidant is a chain terminator consisting of L-methionine, L-cysteine, sodium thiosulfate and BHT.

“Agonists” of IGF-I are compounds, including peptides, which are capable of increasing serum and tissue levels of IGF, especially IGF-I, in a mammal and particularly in a human. See, for example, U.S. Pat. No. 6,251,865 for IGF agonist molecules.

“Lyophilization” refers to a process in which water is removed from a product after it is frozen and placed under a vacuum, allowing the ice to change directly from solid to vapor without passing through a liquid phase. “Lyophilized formulation” refers to a formulation that has been lyophilized, for example to improve stability during storage and increase shelf life and is typically reconstituted prior to administration.

“Stability” refers to the extent to which a drug product retains the characteristics and properties possessed at manufacture during its period of storage and use. Stability includes aspects with regard to its formulation, the stability of IGF-1 and IGFBP (such as IGFBP3) ingredients, the integrity and stability of the (IGF-1/IGFBP3) complex, container and closure, manufacturing and processing conditions, packaging components, storage and shipping conditions, temperature, light, and humidity, and the anticipated duration and conditions of pharmacy shelf-life and patient use. In some embodiments, stability refers to reduced amounts of undesirable species including oxidized species, reduced high molecular weight species that result in aggregation and/or reduced low molecular weight species represented by degradation products. In some embodiments, stability refers to less than 10% of undesirable species. In some embodiments, stability refers to less than 5% of undesirable species. In some embodiments, stability refers to less than 2% of undesirable species.

### IGF-1/IGFBP-3

IGF-1 or an agonist or an analog thereof may be used to practice the present invention. IGF-I is a well-known regulator of postnatal growth and metabolism. See, Baker J, Liu J P, Robertson E J, Efstratiadis A. It has a molecular weight of approximately 7.5 kilodaltons (Kd). Most circulating IGF is bound to the IGF-binding protein, and more particularly to IGFBP-3. IGF-I may be measured in blood serum to diagnose abnormal growth-related conditions.

Typically, a therapeutic composition suitable for treatment of the diseases described herein, including for example CLD, contains an IGF-1 and an IGF-1 binding protein such as IGF binding-proteins (IGFBPs). At least six distinct IGF binding-proteins (IGFBPs) have been identified in various tissues and body fluids. In some embodiments, a suitable therapeutic composition according to the present invention contains IGF-1 and IGFBP-3. IGF-1 and IGFBP-3 may be used as a protein complex or separately. In some embodiments, IGF-1 and IGFBP (such as IGFBP-3) are complexed in equimolar amounts. In some embodiments, a therapeutic composition comprises mecasermin rinfabate. In some embodiments, a therapeutic composition comprises mecasermin rinfabate and a surfactant. In some embodiments, a therapeutic composition comprises mecasermin rinfabate, a surfactant and an antioxidant. In some embodiments, a therapeutic composition comprises mecasermin rinfabate and polysorbate 20. In some embodiments, a therapeutic composition comprises mecasermin rinfabate and polysorbate 20 and an antioxidant. In some embodiments, a therapeutic composition comprises mecasermin rinfabate, polysorbate 20 and methionine. In some embodiments, a therapeutic composition comprises mecasermin rinfabate, polysorbate 20 and sodium thiosulfate. In some embodiments, a therapeutic composition comprises mecasermin rinfabate, polysorbate 80 and an antioxidant. In some embodiments, a therapeutic composition comprises mecasermin rinfabate, polysorbate 80 and methionine. In some embodiments, a therapeutic composition comprises mecasermin rinfabate, polysorbate 80 and sodium thiosulfate.

IGF-I and IGF-I binding proteins such as IGFBP-3 may be purified from natural sources or produced by recombinant means. For instance, purification of IGF-I from human serum is well known in the art (Rinderknecht et al. (1976) Proc. Natl. Acad. Sci. USA 73:2365-2369). Production of IGF-I by recombinant processes is shown in EP 0128733, published in December of 1984. IGFBP-3 may be purified from natural sources using a process such as that shown by Baxter et al. (1986, Biochem. Biophys. Res. Comm. 139:1256-1261). Alternatively, IGFBP-3 may be synthesized recombinantly as discussed by Sommer et al., pp. 715-728, Modern Concepts Of Insulin-Like Growth Factors (E. M. Spencer, ed., Elsevier, N.Y., 1991). Recombinant IGFBP-3 binds IGF-I in a 1:1 molar ratio (equimolar amounts).

### Pharmaceutical Composition and Therapeutic Use

In one aspect, provided herein is a composition comprising an isolated protein complex comprising insulin-like growth factor 1 (IGF-1) and insulin-like growth factor binding protein (such as IGFBP-3), for example at a molar ratio of 1:1, wherein the composition comprises an antioxidant, and wherein the amount of oxidized IGF-1 species does not increase more than 50% upon storage at a temperature of about 25° C. after three months.

The formulations of the present invention are used in the treatment of patients suffering from diseases and complications of prematurity, for example, Intraventricular Hemorrhage (IVH), Bronchopulmonary Dysplasia (BPD) or Chronic Lung Disease (CLD), such as for example, CLD associated with prematurity. For example, the present invention may be used to treat a premature infant who is suffering from CLD or complication associated with CLD. In some embodiments, the present invention may be used to treat an infant who is prematurely born by at least 1 week, 2 weeks, 3 weeks, 4 weeks, 1 month, 2 months, or 3 months. In some embodiments, the present invention may be used to treat an extremely premature infant.

In one embodiment of the invention, IGF-I is administered in combination with IGF binding protein capable of binding IGF-I, and a surfactant. In some embodiments, the IGF binding protein capable of binding IGF-I is IGF binding protein 3 (IGFBP-3). In some embodiments, the composition comprises a surfactant selected from polysorbate 20 or polysorbate 80. In some embodiments, the composition further comprises an antioxidant selected from methionine or sodium thiosulfate.

A composition comprising equimolar amounts of IGF-I and IGF-binding protein 3 may be used. In some embodiments, the IGF-I and IGF binding protein 3 are complexed prior to administration. The complex may be formed by mixing approximately equimolar amounts of IGF-I and IGF binding protein 3 dissolved in physiologically compatible carriers such as normal saline, or phosphate buffered saline solution. In some embodiments, a concentrated solution of recombinant human IGF-I and a concentrated solution of recombinant human IGF binding protein 3 are mixed together for a sufficient time to form an equimolar complex. In some embodiments, recombinant human IGF-I and recombinant human IGF binding protein 3 are combined to form a complex during purification as described in WO96/40736.

### Antioxidants

Oxidation of protein residues arises from a number of different sources. Beyond the addition of specific antioxidants, the prevention of oxidative protein damage involves the careful control of a number of factors throughout the manufacturing process and storage of the product such as atmospheric oxygen, temperature, light exposure, and chemical contamination. The invention therefore contemplates the use of the pharmaceutical antioxidants including, without limitation, reducing agents, oxygen/free-radical scavengers, or chelating agents.

Antioxidants are compounds included in the formulation that prevent or reduce the formation of oxidized species. Oxidation reactions transfer electrons from a substance to an oxidizing agent. During this process, some free-radicals are produced, which starts chain reactions that damage animal cells. Without wishing to be bound by any particular theory, it is contemplated that antioxidants slow down these chain reactions by removing free-radical intermediates and eventually inhibit other oxidation reactions by being oxidized themselves. Antioxidants often play the role of a reducing agent, e.g., thiols or polyphenols.

Antioxidants in therapeutic protein formulations are, in one aspect, water-soluble and remain active throughout the product shelf-life. Reducing agents and oxygen/free-radical scavengers work by ablating active oxygen species in solution. Chelating agents such as EDTA are effective by binding trace metal contaminants that promote free-radical formation.

In addition to the effectiveness of various excipients to prevent protein oxidation, the potential for the antioxidants themselves to induce other covalent or physical changes to the protein is of concern. For example, reducing agents can cause disruption of intramolecular disulfide linkages, which can lead to disulfide shuffling. In the presence of transition metal ions, ascorbic acid and EDTA have been shown to promote methionine oxidation in a number of proteins and peptides (Akers M J, and Defelippis M R. Peptides and Proteins as Parenteral Solutions. In: Pharmaceutical Formulation Development of Peptides and Proteins. Sven Frokjaer, Lars Hovgaard, editors. Pharmaceutical Science. Taylor and Francis, UK (1999)); Fransson J. R., J. Pharm. Sci. 86 (9): 4046-1050 (1997); Yin J, et al., Pharm Res., 21 (12): 2377-83 (2004)). Sodium thiosulfate has been reported to reduce the levels of light and temperature induced methionine-oxidation in rhuMab HER2; however, the formation of a thiosulfate-protein adduct has also been reported (Lam X M, Yang J Y, et al., J Pharm Sci. 86 (11): 1250-5 (1997)). Selection of an appropriate antioxidant is made according to the specific stresses and sensitivities of the protein. Antioxidants contemplated in certain aspects include, without limitation, reducing agents and oxygen/free-radical scavengers, EDTA, and sodium thiosulfate.

In some embodiments, the antioxidant is selected from a reducing agent, free radical scavenger, chelating agent, chain terminator, nitrogen, etc. Methionine has been observed to be effective against a number of oxidative stresses (Lam X M, et al., J Pharm Sci., 86 (11): 1250-5 (1997)).

In some embodiments, the antioxidant is a reducing agent selected from a group consisting of L-methionine, sodium metabisulfite, sodium thiosulfate and glutathione. In some embodiments, the antioxidant is a free radical scavenger selected from a group consisting of L-histidine, L-ascorbic acid, catalase and platinum. In some embodiments, the antioxidant is a chelating agent selected from a group consisting of sodium salt of EDTA dihydrate, diethylenetriaminepentaacetic acid (DTPA) and citric acid.

In some embodiments, the antioxidant is a chain terminator consisting of L-methionine, L-cysteine, sodium thiosulfate and BHT. In some embodiments, oxidation is reduced by repeated pull vacuum and addition or operation under nitrogen.

In some embodiments, the composition comprises 0.05 mM methionine, 0.10 mM methionine, 0.15 mM methionine, 0.20 mM methionine, 0.25 mM methionine, 0.30 mM methionine, 0.35 mM methionine, 0.40 mM methionine, 0.45 mM methionine, 0.50 mM methionine, 0.55 mM methionine, 0.60 mM methionine, 0.65 mM methionine, 0.70 mM methionine, 0.75 mM methionine, 0.80 mM methionine, 0.85 mM methionine, 0.90 mM methionine, 0.95 mM methionine, or 1 mM methionine.

In some embodiments, the composition comprises 1 mM methionine, 2 mM methionine, 3 mM methionine, 4 mM methionine, 5 mM methionine, 6 mM methionine, 7 mM methionine, 8 mM methionine, 9 mM methionine, 10 mM methionine, 11 mM methionine, 12 mM methionine, 13 mM methionine, 14 mM methionine, 15 mM methionine, 16 mM methionine, 17 mM methionine, 18 mM methionine, 19 mM methionine, or 20 mM methionine.

In some embodiments, the composition comprises 25 mM methionine, 30 mM methionine, 35 mM methionine, 40 mM methionine, 45 mM methionine, 50 mM methionine, 55 mM methionine, 60 mM methionine, 65 mM methionine, 70 mM methionine, 75 mM methionine, 80 mM methionine, 85 mM methionine, 90 mM methionine, 95 mM methionine, or 100 mM methionine.

In some embodiments, the composition comprises 0.05 mM sodium thiosulfate, 0.10 mM sodium thiosulfate, 0.15 mM sodium thiosulfate, 0.20 mM sodium thiosulfate, 0.25 mM sodium thiosulfate, 0.30 mM sodium thiosulfate, 0.35 mM sodium thiosulfate, 0.40 mM sodium thiosulfate, 0.45 mM sodium thiosulfate, 0.50 mM sodium thiosulfate, 0.55 mM sodium thiosulfate, 0.60 mM sodium thiosulfate, 0.65 mM sodium thiosulfate, 0.70 mM sodium thiosulfate, 0.75 mM sodium thiosulfate, 0.80 mM sodium thiosulfate, 0.85 mM sodium thiosulfate, 0.90 mM sodium thiosulfate, 0.95 mM sodium thiosulfate, or 1 mM sodium thiosulfate.

In some embodiments, the composition comprises 1 mM sodium thiosulfate, 2 mM sodium thiosulfate, 3 mM sodium thiosulfate, 4 mM sodium thiosulfate, 5 mM sodium thiosulfate, 6 mM sodium thiosulfate, 7 mM sodium thiosulfate, 8 mM sodium thiosulfate, 9 mM sodium thiosulfate, 10 mM sodium thiosulfate, 11 mM sodium thiosulfate, 12 mM sodium thiosulfate, 13 mM sodium thiosulfate, 14 mM sodium thiosulfate, 15 mM sodium thiosulfate, 16 mM sodium thiosulfate, 17 mM sodium thiosulfate, 18 mM sodium thiosulfate, 19 mM sodium thiosulfate, or 20 mM sodium thiosulfate.

In some embodiments, the composition comprises 25 mM sodium thiosulfate, 30 mM sodium thiosulfate, 35 mM sodium thiosulfate, 40 mM sodium thiosulfate, 45 mM sodium thiosulfate, 50 mM sodium thiosulfate, 55 mM sodium thiosulfate, 60 mM sodium thiosulfate, 65 mM sodium thiosulfate, 70 mM sodium thiosulfate, 75 mM sodium thiosulfate, 80 mM sodium thiosulfate, 85 mM sodium thiosulfate, 90 mM sodium thiosulfate, 95 mM sodium thiosulfate, or 100 mM sodium thiosulfate.

In some embodiments, the composition further comprises a buffer comprising sodium acetate, acetic acid and/or sodium chloride. In some embodiments, the composition further comprises a buffer comprising sodium acetate or acetic acid. In some embodiments, the composition further comprises a buffer comprising sodium chloride.

In some embodiments, the sodium acetate or acetic acid is at a concentration of between about 10 and 100 mM. In some embodiments, the sodium acetate or acetic acid is at a concentration of about 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM or 100 mM. In some embodiments, the sodium acetate or acetic acid is at a concentration of about 50 mM.

In some embodiments, the sodium chloride is at a concentration of about 20 mM and 200 mM. In some embodiments, the sodium chloride is at a concentration of about 20 mM, about 25 mM, 30 mM, 35 mM, 40 mM, 45 mM, 50 mM, 55 mM, 60 mM, 65 mM, 70 mM, 75 mM, 80 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, or about 200 mM. In some embodiments, the sodium chloride is at a concentration of about 105 mM.

In some embodiments, the composition is at a pH between about 5.0 and 6.0. In some embodiments, the pH is about 5.0, about 5.2, about 5.4, about 5.6, about 5.8, about 6.0, about 6.2, about 6.4, about 6.6, about 6.8 or about 7.0. In some embodiments, the composition is at a pH of about 5.1, about 5.3, about 5.5, about 5.7, about 5.9, about 6.1, about 6.3, about 6.5, about 6.7, or about 6.9. In some embodiments, the composition is at a pH of about 5.5.

### Lyophilization

In one aspect, the formulations comprising an IGF-1/IGFBP (such as IGFBP-3) are lyophilized prior to administration. A lyophilization cycle is, in one aspect, composed of three steps: freezing, primary drying, and secondary drying. In the freezing step, the solution is cooled to initiate ice formation. Furthermore, this step induces the crystallization of the bulking agent. The ice sublimes in the primary drying stage, which is conducted by reducing chamber pressure below the vapor pressure of the ice, using a vacuum and introducing heat to promote sublimation. Finally, adsorbed or bound water is removed at the secondary drying stage under reduced chamber pressure and at an elevated shelf temperature. The process produces a material known as a lyophilized cake. Thereafter the cake can be reconstituted with either sterile water or suitable diluent for injection.

The lyophilization cycle not only determines the final physical state of excipients but also affects other parameters such as reconstitution time, appearance, stability and final moisture content. The composition structure in the frozen state proceeds through several transitions (e.g., glass transitions, wettings, and crystallizations) that occur at specific temperatures and the structure may be used to understand and optimize the lyophilization process. The glass transition temperature (Tg and/or Tg′) can provide information about the physical state of a solute and can be determined by differential scanning calorimetry (DSC). Tg and Tg′ are an important parameter that must be taken into account when designing the lyophilization cycle. For example, Tg′ is important for primary drying. Furthermore, in the dried state, the glass transition temperature provides information on the storage temperature of the final product. For example, in some embodiments, a lyophilization run is carried out under a lyophilization cycle consisting of a primary drying temperature of −25° C. and a secondary drying temperature of 25° C. using a lyostar lyophilizer. In some embodiments, a lyophilization run is carried out under a lyophilization cycle consisting of a primary drying temperature of −20° C. and a secondary drying temperature of 25° C. using a lyostar lyophilizer.

### Formulations and Excipients

Excipients are additives that impart or enhance the stability and delivery of a drug product (e.g., IGF-1/IGFBP (such as IGFBP-3) protein complex). Excipients are an integral component of a formulation and therefore need to be safe and well tolerated by patients. For protein drugs, the choice of excipients is particularly important because they can affect both efficacy and immunogenicity of the drug. Hence, protein formulations need to be developed with appropriate selection of excipients that afford suitable stability, safety, and marketability.

In some embodiments, a pharmaceutical composition is provided comprising the composition described herein and one or more suitable pharmaceutical excipients.

In some embodiments, the composition comprises polysorbate 20. In some embodiments, the polysorbate 20 surfactant is at a concentration of between about 0.001% to 2.4% v/v. In some embodiments, the polysorbate 20 surfactant is at a concentration of between about 0.2% to 0.4% v/v. In some embodiments, polysorbate 20 surfactant is at a concentration of about 0.001%, 0.005%, 0.01%, 0.02%, 0.03%, 0.05%, 0.10%, 0.15%, 0.2%, 0.5%, 0.7%, 1.0%, 1.2%, 1.5%, 1.8%, 2.0%, 2.2% or 2.4% v/v. In some embodiments, the polysorbate 20 surfactant is at a concentration of about 0.0025%, about 0.005%, or about 0.0075% v/v. In some embodiments, the polysorbate 20 is at a concentration of 0.005%.

In some embodiments, the composition comprises polysorbate 80. In some embodiments, the polysorbate 80 surfactant is at a concentration of between about 0.001% to 2.4% v/v. In some embodiments, the polysorbate 80 surfactant is at a concentration of between about 0.2% to 0.4% v/v. In some embodiments, polysorbate 80 surfactant is at a concentration of about 0.001%, 0.005%, 0.01%, 0.02%, 0.03%, 0.05%, 0.10%, 0.15%, 0.2%, 0.5%, 0.7%, 1.0%, 1.2%, 1.5%, 1.8%, 2.0%, 2.2% or 2.4% v/v. In some embodiments, the polysorbate 80 surfactant is at a concentration of about 0.0025%, about 0.005%, or about 0.0075% v/v. In some embodiments, the polysorbate 80 is at a concentration of 0.005%.

A lyophilized formulation is, in one aspect, at least comprised of one or more of a buffer, a bulking agent, a stabilizer, and surfactant. An appropriate buffering agent is included to maintain the formulation within stable zones of pH during lyophilization.

A challenge in developing protein formulations for proteins is stabilizing the product against the stresses of manufacturing, shipping and storage. The role of formulation excipients is to provide stabilization against these stresses. Excipients are also employed to reduce viscosity of high concentration protein formulations in order to enable their delivery and enhance patient convenience.

In general, excipients can be classified on the basis of the mechanisms by which they stabilize proteins against various chemical and physical stresses. Some excipients are used to alleviate the effects of a specific stress or to regulate a particular susceptibility of a specific protein. Other excipients have more general effects on the physical and covalent stabilities of proteins. The excipients described herein are organized either by their chemical type or their functional role in formulations. Brief descriptions of the modes of stabilization are provided when discussing each excipient type. The amount and type of a salt to be included in a biopharmaceutical formulation of the invention is selected based on the desired osmolality (i.e., isotonic, hypotonic or hypertonic) of the final solution as well as the amounts and osmolality of other components to be included in the formulation.

By way of example, inclusion of about 5% sorbitol can achieve isotonicity while about 9% of a sucrose excipient is needed to achieve isotonicity. Selection of the amount or range of concentrations of one or more excipients that can be included within a biopharmaceutical formulation of the invention has been exemplified above by reference to salts, polyols and sugars. However, those skilled in the art will understand that the considerations described herein and further exemplified by reference to specific excipients are equally applicable to all types and combinations of excipients including, for example, salts, amino acids, other tonicity agents, surfactants, stabilizers, bulking agents, cryoprotectants, lyoprotectants, anti-oxidants, metal ions, chelating agents and/or preservatives.

Further, where a particular excipient is reported in molar concentration, those skilled in the art will recognize that the equivalent percent (%) w/v (e.g., (grams of substance in a solution sample/mL of solution)×100%) of solution is also contemplated.

In some embodiments, concentration of a bulking agent may be lowered where, e.g., there is a high protein concentration or where, e.g., there is a high stabilizing agent concentration. In some embodiments, to maintain the isotonicity of a particular formulation in which there is no bulking agent, the concentration of a stabilizing agent would be adjusted accordingly (i.e., a “tonicifying” amount of stabilizer would be used).

A comparison of the excipient components contemplated for liquid and lyophilized protein formulations is provided in Table A.

TABLE A
Excipient components of lyophilized protein formulations

| Excipient | |
|---|---|
| component | Function in lyophilized formulation |
| Buffer | Maintain pH of formulation during lyophilization |
| | and upon reconstitution |
| Tonicity agent/ | Stabilizers include cryo and lyoprotectants e.g. |
| stabilizer | polyols, sugars, polymers. Cryoprotectants protect |
| | proteins from freezing stresses; lyoprotectants |
| | stabilize proteins in the freeze-dried state. |
| Bulking agent | To enhance product; provides structural strength |
| | to the lyo cake e.g. mannitol and glycine. |
| Surfactant | Helps minimize aggregation during lyo process; |
| | reduces reconstitution times e.g. polysorbate 20 |
| | and 80 |
| Antioxidant | Molecular reactions in the lyo cake are highly |
| | retarded, so while these are added to liquid |
| | formulations, not needed in a lyo formulation. |
| Metal ions/ | A specific metal ion may be included as co-factor |
| chelating agent | or where a metal is required for protease activity. |
| | Chelating agents are not required in lyo formulations. |
| Preservative | For multi-dose formulations only. |
| | Provides protection against microbial growth |
| | Is included in the reconstitution diluent |

### Buffers and Buffering Agents

The stability of a pharmacologically active protein formulation is usually observed to be maximal in a narrow pH range. This pH range of optimal stability needs to be identified early during pre-formulation studies. Several approaches, such as accelerated stability studies and calorimetric screening studies, are useful in this endeavor. Once a formulation is finalized, the protein must be manufactured and maintained throughout its shelf-life. Hence, buffering agents are almost always employed to control pH in the formulation.

The buffer capacity of the buffering species is maximal at a pH equal to the pKa and decreases as pH increases or decreases away from this value. Ninety percent of the buffering capacity exists within one pH unit of its pKa. Buffer capacity also increases proportionally with increasing buffer concentration. When selecting a buffer, the buffer species and its concentration need to be defined based on its pKa and the desired formulation pH. It is also important is to ensure that the buffer is compatible with the protein and other formulation excipients, and does not catalyze any degradation reactions. Another aspect to be considered is the sensation of stinging and irritation the buffer may induce upon administration. For example, citrate is known to cause stinging upon injection, especially when administered via the subcutaneous (SC) or intramuscular (IM) routes, where the drug solution remains at the site for a relatively longer period of time than when administered by the IV route where the formulation gets diluted rapidly into the blood upon administration. For formulations that are administered by direct IV infusion, the total amount of buffer (and any other formulation component) needs to be monitored, and potassium ions administered in the form of the potassium phosphate buffer, should be monitored for cardiovascular effects.

Buffers for lyophilized formulations need additional consideration. Some buffers like sodium phosphate can crystallize out of the protein amorphous phase during freezing resulting in shifts in pH. Other common buffers such as acetate and imidazole may sublime or evaporate during the lyophilization process, thereby shifting the pH of formulation during lyophilization or after reconstitution.

The buffer system present in the compositions is selected to be physiologically compatible and to maintain a desired pH of the pharmaceutical formulation. In one embodiment, the pH of the solution is between pH 2.0 and pH 12.0. For example, the pH of the solution may be 2.0, 2.3, 2.5, 2.7, 3.0, 3.3, 3.5, 3.7, 4.0, 4.3, 4.5, 4.7, 5.0, 5.3, 5.5, 5.7, 6.0, 6.3, 6.5, 6.7, 7.0, 7.3, 7.5, 7.7, 8.0, 8.3, 8.5, 8.7, 9.0, 9.3, 9.5, 9.7, 10.0, 10.3, 10.5, 10.7, 11.0, 11.3, 11.5, 11.7, or 12.0.

The pH buffering compound may be present in any amount suitable to maintain the pH of the formulation at a predetermined level. In one embodiment, the pH buffering concentration is between 0.1 mM and 500 mM (1 M). For example, it is contemplated that the pH buffering agent is at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM.

Exemplary pH buffering agents used to buffer the formulation as set out herein include, but are not limited to organic acids, glycine, histidine, glutamate, succinate, phosphate, acetate, citrate, Tris, HEPES, and amino acids or mixtures of amino acids, including, but not limited to aspartate, histidine, and glycine. In one embodiment of the present invention, the buffering agent is citrate.

### Stabilizers and Bulking Agents

In one aspect, a stabilizer (or a combination of stabilizers) is added to prevent or reduce storage-induced aggregation and chemical degradation. A hazy or turbid solution upon reconstitution indicates that the protein has precipitated or at least aggregated. The term “stabilizer” refers to an excipient capable of preventing aggregation or physical degradation, including chemical degradation (for example, autolysis, deamidation, oxidation, etc.) in an aqueous state. Stabilizers contemplated include, but are not limited to, sucrose, trehalose, mannose, maltose, lactose, glucose, raffinose, cellobiose, gentiobiose, isomaltose, arabinose, glucosamine, fructose, mannitol, sorbitol, glycine, arginine HCL, poly-hydroxy compounds, including polysaccharides such as dextran, starch, hydroxyethyl starch, cyclodextrins, N-methyl pyrollidene, cellulose and hyaluronic acid, sodium chloride. In the present formulations, the stabilizer is incorporated in a concentration of about 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, 900, or 1000 mM. In one embodiment, mannitol and trehalose are used as stabilizing agents.

In further embodiments, the formulations also comprise appropriate amounts of bulking and osmolarity regulating agents. Bulking agents include, for example, mannitol, glycine, sucrose, polymers such as dextran, polyvinylpyrolidone, carboxymethylcellulose, lactose, sorbitol, trehalose, or xylitol. In one embodiment, the bulking agent is mannitol. The bulking agent is incorporated in a concentration of about 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, 900, or 1000 mM.

### Surfactants

Proteins have a high propensity to interact with surfaces making them susceptible to adsorption and denaturation at air-liquid, vial-liquid, and liquid-liquid (silicone oil) interfaces. This degradation pathway has been observed to be inversely dependent on protein concentration and results in either the formation of soluble and insoluble protein aggregates or the loss of protein from solution via adsorption to surfaces. In addition to container surface adsorption, surface-induced degradation is exacerbated with physical agitation, as would be experienced during shipping and handling of the product.

Surfactants are commonly used in protein formulations to prevent surface-induced degradation. Surfactants are amphipathic molecules with the capability of out-competing proteins for interfacial positions. Hydrophobic portions of the surfactant molecules occupy interfacial positions (e.g., air/liquid), while hydrophilic portions of the molecules remain oriented towards the bulk solvent. At sufficient concentrations (typically around the detergent's critical micellar concentration), a surface layer of surfactant molecules serve to prevent protein molecules from adsorbing at the interface. Thereby, surface-induced degradation is minimized. Surfactants contemplated herein include, without limitation, fatty acid esters of sorbitan polyethoxylates, i.e. polysorbate 20 and polysorbate 80. The two differ only in the length of the aliphatic chain that imparts hydrophobic character to the molecules, C-12 and C-18, respectively. Accordingly, polysorbate 80 is more surface-active and has a lower critical micellar concentration than polysorbate 20.

Detergents affect the thermodynamic conformational stability of proteins. Hydrophobic tails of the detergent molecules that can engage in specific binding with partially or wholly unfolded protein states.

These types of interactions could cause a shift in the conformational equilibrium towards the more expanded protein states (i.e. increasing the exposure of hydrophobic portions of the protein molecule in complement to binding polysorbate). Alternatively, if the protein native state exhibits some hydrophobic surfaces, detergent binding to the native state may stabilize that conformation.

Another aspect of polysorbates is that they are inherently susceptible to oxidative degradation. Often, as raw materials, they contain sufficient quantities of peroxides to cause oxidation of protein residue side-chains, especially methionine. The potential for oxidative damage arising from the addition of stabilizer emphasizes the point that the lowest effective concentrations of excipients should be used in formulations. For surfactants, the effective concentration for a given protein will depend on the mechanism of stabilization.

Surfactants are also added in appropriate amounts to prevent surface related aggregation phenomenon during freezing and drying. Thus, exemplary surfactants include, without limitation, anionic, cationic, nonionic, zwitterionic, and amphoteric surfactants including surfactants derived from naturally-occurring amino acids. Anionic surfactants include, but are not limited to, sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt. Cationic surfactants include, but are not limited to, benzalkonium chloride or benzethonium chloride, cetylpyridinium chloride monohydrate, and hexadecyltrimethylammonium bromide. Zwitterionic surfactants include, but are not limited to, CHAPS, CHAPSO, SB3-10, and SB3-12. Non-ionic surfactants include, but are not limited to, digitonin, Triton X-100, Triton X-114, TWEEN-20, and TWEEN-80. Surfactants also include, but are not limited to lauromacrogol 400, polyoxyl 40 stearate, polyoxyethylene hydrogenated castor oil 10, 40, 50 and 60, glycerol monostearate, polysorbate 40, 60, 65 and 80, soy lecithin and other phospholipids such as dioleyl phosphatidyl choline (DOPC), dimyristoylphosphatidyl glycerol (DMPG), dimyristoylphosphatidyl choline (DMPC), and (dioleyl phosphatidyl glycerol) DOPG; sucrose fatty acid ester, methyl cellulose and carboxymethyl cellulose. Compositions comprising these surfactants, either individually or as a mixture in different ratios, are therefore further provided. In one embodiment of the present invention, the surfactant is TWEEN-80. In the present formulations, the surfactant is incorporated in a concentration of about 0.01 to about 0.5 g/L. In some embodiments of the formulations provided, the surfactant concentration is 0.005, 0.01, 0.02, 0.03, 0.05, 0.06, 0.07, 0.08, 0.09, 0.1, 0.2, 0.3, 0.4, 0.5, 0.6, 0.7, 0.8, 0.9 or 1.0 g/L. In some embodiments, the surfactant is polysorbate 20. In some embodiments, the surfactant is polysorbate 80. In some embodiments, the surfactant is 0.005% polysorbate 20. In some embodiments, the surfactant is 0.005% polysorbate 80.

### Salts

Salts are often added to increase the ionic strength of the formulation, which can be important for protein solubility, physical stability, and isotonicity. Salts can affect the physical stability of proteins in a variety of ways. Ions can stabilize the native state of proteins by binding to charged residues on the protein's surface. Alternatively, salts can stabilize the denatured state by binding to peptide groups along the protein backbone (—CONH—). Salts can also stabilize the protein native conformation by shielding repulsive electrostatic interactions between residues within a protein molecule. Salts in protein formulations can also shield attractive electrostatic interactions between protein molecules that can lead to protein aggregation and insolubility. In formulations provided, the salt concentration is between 0.1, 1, 10, 20, 30, 40, 50, 80, 100, 120, 150, 200, 300, and 500 mM.

### Other Common Excipient Components

### Amino Acids

Amino acids have found versatile use in protein formulations as buffers, bulking agents, stabilizers and antioxidants. Thus, in one aspect histidine and glutamic acid are employed to buffer protein formulations in the pH range of 5.5-6.5 and 4.0-5.5 respectively. The imidazole group of histidine has a pKa=6.0 and the carboxyl group of glutamic acid side chain has a pKa of 4.3 which makes these amino acids suitable for buffering in their respective pH ranges. Glutamic acid is particularly useful in such cases. Histidine is commonly found in marketed protein formulations, and this amino acid provides an alternative to citrate, a buffer known to sting upon injection. Interestingly, histidine also has a stabilizing effect, with respect to aggregation when used at high concentrations in both liquid and lyophilized presentations, and reduces the viscosity of a high protein concentration formulation.

In various aspects, formulations are provided which include one or more of the amino acids glycine, proline, serine, arginine and alanine which have been shown to stabilize proteins by the mechanism of preferential exclusion. Glycine is also a commonly used bulking agent in lyophilized formulations. Arginine has been shown to be an effective agent in inhibiting aggregation and has been used in both liquid and lyophilized formulations. In some embodiments, the amino acid concentration is between 0.1, 1, 10, 20, 30, 40, 50, 80, 100, 120, 150, 200, 300, and 500 mM.

### Metal Ions

In general, transition metal ions are undesired in protein formulations because they can catalyze physical and chemical degradation reactions in proteins. However, specific metal ions are included in formulations when they are co-factors to proteins and in suspension formulations of proteins where they form coordination complexes (e.g., zinc suspension of insulin). Further, magnesium ions (10-120 mM) has been proposed to inhibit the isomerization of aspartic acid to isoaspartic acid.

Two examples where metal ions confer stability or increased activity in proteins are human deoxyribonuclease (rhDNase, Pulmozyme®), and Factor VIII. In the case of rhDNase, Ca+2 ions (up to 100 mM) increased the stability of the enzyme through a specific binding site. In fact, removal of calcium ions from the solution with EGTA caused an increase in deamidation and aggregation. However, this effect was observed only with Ca+2 ions; other divalent cations Mg+2, Mn+2 and Zn+2 were observed to destabilize rhDNase. Similar effects were observed in Factor VIII. Ca+2 and Sr+2 ions stabilized the protein while others like Mg+2, Mn+2 and Zn+2, Cu+2 and Fe+2 destabilized the enzyme (Fatouros, A., et al., Int. J. Pharm., 155, 121-131 (1997). In a separate study with Factor VIII, a significant increase in aggregation rate was observed in the presence of Al+3 ions (Derrick T S, et al., J. Pharm. Sci., 93 (10): 2549-57 (2004)). Other excipients like buffer salts are often contaminated with Al+3 ions and this illustrates the need to use excipients of appropriate quality in formulated products.

### Preservatives

Preservatives are necessary when developing multi-use parenteral formulations that involve more than one extraction from the same container. Their primary function is to inhibit microbial growth and ensure product sterility throughout the shelf-life or use of the drug product. Commonly used preservatives include, without limitation, benzyl alcohol, phenol and m-cresol. Although preservatives have a long history of use, the development of protein formulations that includes preservatives can be challenging. Preservatives almost always have a destabilizing effect (aggregation) on proteins, and this has become a major factor in limiting their use in multi-dose protein formulations (Roy S, et al., J Pharm Sci., 94 (2): 382-96 (2005)). To date, most protein drugs have been formulated for single-use only. When multi-dose formulations are possible, they have the added advantage of enabling patient convenience, and increased marketability. In the case of human growth hormone (hGH) where the development of preserved formulations has led to commercialization of more convenient, multi-use injection pens, for example, Norditropin® (liquid, Novo Nordisk), Nutropin AQ® (liquid, Genentech) and Genotropin (lyophilized—dual chamber cartridge, Pharmacia & Upjohn) contain phenol while Somatrope® (Eli Lilly) is formulated with m-cresol.

Several aspects need to be considered during the formulation development of preserved dosage forms. The effective preservative concentration in the drug product must be optimized. This requires testing a given preservative in the dosage form with concentration ranges that confer anti-microbial effectiveness without compromising protein stability. For example, three preservatives were successfully screened in the development of a liquid formulation for interleukin-1 receptor (Type I), using differential scanning calorimetry (DSC). The preservatives were rank ordered based on their impact on stability at concentrations commonly used in marketed products.

Development of liquid formulations containing preservatives are more challenging than lyophilized formulations. Freeze-dried products can be lyophilized without the preservative and reconstituted with a preservative containing diluent at the time of use. This shortens the time for which a preservative is in contact with the protein significantly minimizing the associated stability risks. With liquid formulations, preservative effectiveness and stability have to be maintained over the entire product shelf-life (˜18-24 months). An important point to note is that preservative effectiveness has to be demonstrated in the final formulation containing the active drug and all excipient components.

Some preservatives can cause injection site reactions, which is another factor that needs consideration when choosing a preservative. In clinical trials that focused on the evaluation of preservatives and buffers in Norditropin, pain perception was observed to be lower in formulations containing phenol and benzyl alcohol as compared to a formulation containing m-cresol (Kappelgaard A. M., Horm Res. 62 Suppl 3:98-103 (2004)). Interestingly, among the commonly used preservative, benzyl alcohol possesses anesthetic properties (Minogue S C, and Sun D A., Anesth Analg., 100 (3): 683-6 (2005)). In various aspects the use of preservatives provide a benefit that outweighs any side effects.

### Methods of Preparation

The present invention further contemplates methods for the preparation of pharmaceutical formulations. The present methods further comprise one or more of the following steps: adding a stabilizing agent as described herein to said mixture prior to lyophilizing, adding at least one agent selected from a bulking agent, an osmolarity regulating agent, and a surfactant, each of which as described herein, to said mixture prior to lyophilization.

The standard reconstitution practice for lyophilized material is to add back a volume of pure water or sterile water for injection (WFI) (typically equivalent to the volume removed during lyophilization), although dilute solutions of antibacterial agents are sometimes used in the production of pharmaceuticals for parenteral administration [Chen, Drug Development and Industrial Pharmacy, 18:1311-1354 (1992)].

The lyophilized material may be reconstituted as an aqueous solution. A variety of aqueous carriers, e.g., sterile water for injection, water with preservatives for multi dose use, or water with appropriate amounts of surfactants (for example, an aqueous suspension that contains the active compound in admixture with excipients suitable for the manufacture of aqueous suspensions). In various aspects, such excipients are suspending agents, for example and without limitation, sodium carboxymethylcellulose, methylcellulose, hydroxypropylmethylcellulose, sodium alginate, polyvinylpyrrolidone, gum tragacanth and gum acacia; dispersing or wetting agents are a naturally-occurring phosphatide, for example and without limitation, lecithin, or condensation products of an alkylene oxide with fatty acids, for example and without limitation, polyoxyethylene stearate, or condensation products of ethylene oxide with long chain aliphatic alcohols, for example and without limitation, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example and without limitation, polyethylene sorbitan monooleate. In various aspects, the aqueous suspensions also contain one or more preservatives, for example and without limitation, ethyl, or n-propyl, p-hydroxybenzoate.

The pharmaceutical compositions of the invention include those suitable for oral, nasal, topical (including buccal and sublingual), or parenteral (including subcutaneous, intramuscular, intravenous and intradermal) administration. The formulations may conveniently be presented in unit dosage form, e.g., a single use bag containing a single dose (for example a dose for 24 hours), tablets and sustained release capsules, and in liposomes, and may be prepared by any methods well known in the art of pharmacy. See, for example, Remington's Pharmaceutical Sciences, Mack Publishing Company, Philadelphia, Pa. (17th ed. 1985). In some embodiments, the composition is administered intravenously.

Such preparative methods include the step of bringing into association with the molecule to be administered ingredients such as the carrier which constitutes one or more accessory ingredients. In general, the compositions are prepared by uniformly and intimately bringing into association the active ingredients with liquid carriers, liposomes or finely divided solid carriers or both, and then if necessary shaping the product.

Compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

In some embodiments, the methods disclosed herein provide for the parenteral administration of IGF-I complexed with IGF binding protein (such as IGFBP-3) in the presence of a surfactant, e.g., 0.005% polysorbate 20 or polysorbate 80 and an antioxidant, e.g. sodium thiosulfate and methionine, to infants in need of such treatment. Parenteral administration includes, but is not limited to, intravenous (IV), intramuscular (IM), subcutaneous (SC), intraperitoneal (IP), intranasal, and inhalant routes. In some embodiments, the IGF-I/IGFBP-3 composition is administered intravenously. IV, IM, SC, and IP administration may be by bolus or infusion, and may also be by slow release implantable device, including, but not limited to pumps, slow release formulations, and mechanical devices. The formulation, route and method of administration, and dosage will depend on the disorder to be treated and the medical history of the patient. Accordingly, in some embodiments, the methods disclosed herein provide for the intravenous administration of IGF-I complexed with IGF binding protein (such as IGFBP-3) in the presence of a surfactant, e.g., 0.005% polysorbate 20 or polysorbate 80 to infants in need of such treatment. In some embodiments, the methods disclosed herein provide for the subcutaneous administration of IGF-I complexed with IGF binding protein 3 in the presence of a surfactant, e.g., 0.005% polysorbate 20 or polysorbate 80, and antioxidant e.g. methionine or sodium thiosulfate to infants in need of such treatment.

In some embodiments, a composition comprising rIGF-1/IGFBP-3 is at a concentration of between about 10 micrograms/mL-100 micrograms/mL. For example, in some embodiments, a composition comprising rIGF-1/IGFBP-3 is at a concentration of between about 45-55 micrograms/mL. In some embodiments, a composition comprising rIGF-1/IGFBP-3 is at a concentration of about 50 micrograms/mL. In some embodiments, a composition comprising rIGF-1/IGFBP-3 at a concentration of about 10-100 micrograms/mL is suitable for intravenous administration. In some embodiments, a composition comprising rIGF-1/IGFBP-3 at a concentration of about 45-55 micrograms/mL is suitable for intravenous administration. In some embodiments, a composition comprising rIGF-1/IGFBP-3 at a concentration of about 50 micrograms/mL is suitable for intravenous administration.

In some embodiments, a composition comprising rIGF-1/IGFBP-3 is at a concentration of between about 1000 micrograms/mL-5000 micrograms/mL. For example, in some embodiments, a composition comprising rIGF-1/IGFBP-3 is at a concentration of between 2000 micrograms/mL-3000 micrograms/mL. In some embodiments, a composition comprising rIGF-1/IGFBP-3 is at a concentration of about 2500 micrograms/mL. In some embodiments, a composition comprising rIGF-1/IGFBP-3 at a concentration of between about 1000-5000 micrograms/mL is suitable for intravenous administration. In some embodiments, a composition comprising rIGF-1/IGFBP-3 at a concentration of between about 2000 micrograms/mL-3000 micrograms/mL is suitable for intravenous administration. In some embodiments, a composition comprising rIGF-1/IGFBP-3 at a concentration of about 2500 micrograms/mL is suitable for intravenous administration.

A pharmaceutical composition according to the present invention may be administered at various doses. For example, a suitable dosage may range from about 100 to 1000 micrograms/kg/24 hours. In some embodiments, a suitable dosage may be or greater than about 100 micrograms/kg/24 hours, 150 micrograms/kg/24 hours, 200 micrograms/kg/24 hours, 250 micrograms/kg/24 hours, 300 micrograms/kg/24 hours, 350 micrograms/kg/24 hours, 400 micrograms/kg/24 hours, 450 micrograms/kg/24 hours, 500 micrograms/kg/24 hours, 550 micrograms/kg/24 hours, 600 micrograms/kg/24 hours, 650 micrograms/kg/24 hours, 700 micrograms/kg/24 hours, 750 micrograms/kg/24 hours, 800 micrograms/kg/24 hours, 850 micrograms/kg/24 hours, 900 micrograms/kg/24 hours, 950 micrograms/kg/24 hours or 1000 micrograms/kg/24 hours. In some embodiments, a suitable dosage is about 250 micrograms/kg/24 hours. In some embodiments, a suitable dosage is about 400 micrograms/kg/24 hours. In some embodiments, a suitable dosage is about 750 micrograms/kg/24 hours. In some embodiments, a suitable dosage is about 1000 micrograms/kg/24 hours. In some embodiments, a pharmaceutical composition according to the invention is administered from the time of birth up to postmenstrual age (PMA) of about 23 to 34 weeks, up to PMA of about 28 to 32 weeks, up to PMA of about 29 weeks plus 6 days.

For parenteral or oral administration, compositions of the complex may be semi-solid or liquid preparations, such as liquids, suspensions, and the like. Physiologically compatible carriers are those that are non-toxic to recipients at the dosages and concentrations employed and are compatible with other ingredients of the formulation. For example, the formulation preferably does not include oxidizing agents and other compounds that are known to be deleterious to polypeptides. Hence, physiologically compatible carriers include, but are not limited to, normal saline, serum albumin, 5% dextrose, plasma preparations, and other protein-containing solutions. Optionally, the carrier may also include detergents or surfactants.

In some embodiments, provided herein is a dry lyophilized powder. In some embodiments, the composition can be reconstituted in sterile pharmaceutical grade water, saline or buffered water.

In some embodiments, a kit is provided for carrying out the methods described herein. In certain embodiments, the kit comprises a composition comprising rIGF-1, rIGFBP-3, a polysorbate surfactant, such as polysorbate 20 or polysorbate 80 and an antioxidant, such as methionine or sodium thiosulfate. In some embodiments, the kit contains the composition described herein in a vial of glass or other suitable material.

In some embodiments, there is also provided an article of manufacture comprising packaging material and a pharmaceutical agent contained within the packaging material. The packaging material comprises a label which indicates that the pharmaceutical may be administered, for a sufficient term at an effective dose, for treating and/or preventing complications associated with preterm birth. The pharmaceutical agent comprises IGF-I, an agonist or an analog thereof together with a pharmaceutically acceptable carrier.

Values and/or features in an Example(s) may be used as basis for amendment to the claims, wherein the value and/or feature is employed without reference to other elements used in the Example i.e. the values may be employee in isolation.

Embodiments referring to “compositions” herein apply equally to other types of disclosure herein, such as methods and use, unless indicated otherwise and vice versa.

“Is” as employed herein means comprising.

In the context of this specification “comprising” is to be interpreted as “including”.

Embodiments of the invention comprising certain features/elements are also intended to extend to alternative embodiments “consisting” or “consisting essentially” of the relevant elements/features.

Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

The background section contains technically relevant details may be used as basis for an amendment.

Individual numerical values in the examples are generically relevant to aspects and embodiments on the invention and therefore can be used as basis for amendment to the claims.

The invention will be further characterized by the following examples which are intended to be exemplary of the invention.

### BRIEF DESCRIPTION OF THE DRAWING

Drawings are for illustration purposes only; not for limitation.

FIG. 1 is a graph that shows the amount of oxidized rhIGF-1 in percent area measured using reverse phased high performance liquid chromatography (RP-HPLC).

FIG. 2 is a graph that shows the amount of rhIGFBP-3 in percent area measured using RP-HPLC.

FIG. 3 is a graph that shows the amount of rhIGF-1 in percent area measured using RP-HPLC.

FIG. 4A is a graph that shows changes in oxidized rhIGF-1 in a formulation of 0.15 mg/mL protein after 3 months of storage by SEC-HPLC assessment in the presence of either no antioxidant, 0.5 mM or 5 mM methionine or 0.5 mM or 5 mM sodium thiosulfate. FIG. 4B is a graph that shows changes in oxidized rhIGF-1 in a formulation of 0.05 mg/mL protein after 3 months of storage by SEC-HPLC assessment in the presence of either no antioxidant, 0.5 mM or 5 mM methionine, or 0.5 mM or 5 mM sodium thiosulfate.

FIG. 5A is a graph that shows changes in the amount of rhIGF-1 in a formulation of 0.15 mg/mL protein after 3 months of storage by SEC-HPLC assessment in the presence of either no antioxidant, 0.5 mM or 5 mM methionine or 0.5 mM or 5 mM sodium thiosulfate. FIG. 5B is a graph that shows changes in the amount of rhIGF-1 in a formulation of 0.05 mg/mL protein after 3 months of storage by SEC-HPLC assessment in the presence of either no antioxidant, 0.5 mM or 5 mM methionine, or 0.5 mM or 5 mM sodium thiosulfate.

FIG. 6A is a graph that shows changes in the amount of rhIGF-BP3 in a formulation of 0.15 mg/mL protein after 3 months of storage by SEC-HPLC assessment in the presence of either no antioxidant, 0.5 mM or 5 mM methionine or 0.5 mM or 5 mM sodium thiosulfate.

FIG. 6B is a graph that shows changes in the amount of rhIGF-BP3 in a formulation of 0.05 mg/mL protein after 3 months of storage by SEC-HPLC assessment in the presence of either no antioxidant, 0.5 mM or 5 mM methionine, or 0.5 mM or 5 mM sodium thiosulfate.

FIG. 7A is an SDS-PAGE gel that shows stability of 150 μg/mL of IGF-1/IGFBP-3 protein after 3 months of storage in the presence of either no antioxidant, methionine or sodium thiosulfate at 2-8° C.

FIG. 7B is an SDS-PAGE gel that shows stability of 50 μg/mL of IGF-1/IGFBP-3 protein after 3 months of storage in the presence of either no antioxidant, methionine or sodium thiosulfate at 2-8° C.

FIG. 7C is an SDS-PAGE gel that shows stability of 150 μg/mL of IGF-1/IGFBP-3 protein after 3 months of storage in the presence of either no antioxidant, methionine or sodium thiosulfate at 2-8° C.

FIG. 7D is an SDS-PAGE gel that shows stability of 50 μg/mL of IGF-1/IGFBP-3 protein after 3 months of storage in the presence of either no antioxidant, methionine or sodium thiosulfate at 2-8° C.

FIG. 8A shows the temperature profile of a lyophilization run that reached pre-determined setpoints.

FIG. 8B shows the pressure profile during the lyophilization run. The Pirani pressure profile showed that sublimation ended after 34 hours of primary drying when the Pirani pressure reached set point of 100 mTorr as measured by the capacitance manometer. Secondary drying led to further desorption of water and the Pirani pressure reached setpoint again during secondary drying.

FIG. 9A shows baseline status of drug product vials from a lyophilization run. FIG. 9B shows the vials after 2 weeks of storage at 60° C. FIG. 9C shows the vials after 4 weeks of storage at 60° C.

FIG. 10A shows the amount of rhIGF-1/rhIGFBP-3 protein complex by SE-UPLC at baseline, after 2 weeks and 4 weeks of storage, compared to liquid formulation.

FIG. 10B shows the amount of high molecular weight species measured by SE-UPLC at baseline, after 2 weeks and 4 weeks of storage, compared to liquid formulation.

FIG. 11A shows the amount of rhIGFBP-3 measured by SE-UPLC at baseline, after 2 weeks of storage, after 4 weeks of storage, compared to liquid formulation.

FIG. 11B shows the amount of rIGF-1 measured by SE-UPLC at baseline, after 2 weeks of storage, after 4 weeks of storage, compared to liquid formulation.

FIG. 11C shows the amount of oxidized rIGF-1 measured by SE-UPLC at baseline, after 2 weeks of storage, after 4 weeks of storage, compared to liquid formulation.

FIG. 12A shows a representative lyophilization run at baseline.

FIG. 12B shows a representative formulation after 4 weeks of storage at 60° C.

FIG. 12C shows a representative formulation after 4 weeks of storage at 60° C.

FIG. 13A shows a representative lyophilization run at baseline.

FIG. 13B shows a representative formulation after 4 weeks of storage at 60° C.

FIG. 13C shows a representative formulation after 4 weeks of storage at 60° C.

FIG. 13D shows a representative formulation after 4 weeks of storage at 60° C.

FIG. 14A shows an SDS PAGE gel of representative lyophilized formulations at baseline.

FIG. 14B shows an SDS PAGE gel of representative lyophilized formulations after 4 weeks of storage at 40° C.

FIG. 15A shows temperature profile of an exemplary lyophilization run.

FIG. 15B shows a pressure profile of an exemplary lyophilization run.

FIG. 16A shows a graph of the amount of percent rhIGF-1/rhIGFBP-3 protein complex from an exemplary lyophilization run.

FIG. 16B shows a graph of the amount of high molecular weight species from an exemplary lyophilization run.

FIG. 17A shows a graph of the amount of rhIGFBP-3 as measured by RP-UPLC at baseline, after 2 weeks of storage, and after 4 weeks of storage, compared to liquid formulation.

FIG. 17B shows a graph of the amount of rhIGF-1 as measured by RP-UPLC at baseline, after 2 weeks of storage, after 4 weeks of storage, compared to liquid formulation.

FIG. 17C shows a graph of the amount of oxidized rhIGF-1 as measured by RP-UPLC at baseline, after 2 weeks of storage, after 4 weeks of storage, compared to liquid formulation.

FIG. 18A shows a graph of a representative lyophilized formulation at baseline.

FIG. 18B shows a graph of a representative formulation after 4 weeks of storage at 40° C.

FIG. 18C shows a graph of a representative formulation after 4 weeks of storage at 40° C.

FIG. 18D shows a graph of a representative formulation after 4 weeks of storage at 40° C.

FIG. 18E shows a graph of a representative formulation after 4 weeks of storage at 40° C.

FIG. 18F shows a graph of a representative formulation after 48 hours of liquid storage at room temperature.

FIG. 19A shows a graph of a representative formulation at baseline.

FIG. 19B shows a graph of a representative formulation after 4 weeks of storage at 40° C.

FIG. 19C shows a graph of a representative formulation after 48 hours of liquid storage at room temperature.

FIG. 20A shows an SDS PAGE gel of representative lyophilized protein formulations at baseline.

FIG. 20B shows an SDS PAGE gel of representative lyophilized protein formulations after 4 weeks of storage at 40° C.

### EXAMPLES

While certain compounds, compositions and methods described herein have been described with specificity in accordance with certain embodiments, the following examples serve only to illustrate the compounds of the invention and are not intended to limit the same.

### Example 1

Measuring Oxidized rIGF-1 by RP-UPLC after the Addition of Antioxidants to an IGF-1/IGFBP-3 Liquid Formulation

This example illustrates the amount of oxidized species in a formulation comprising insulin like growth factor-1/insulin-like growth factor binding protein-3 (rhIGF-1/rhIGFBP-3) complex with and without the addition of antioxidants.

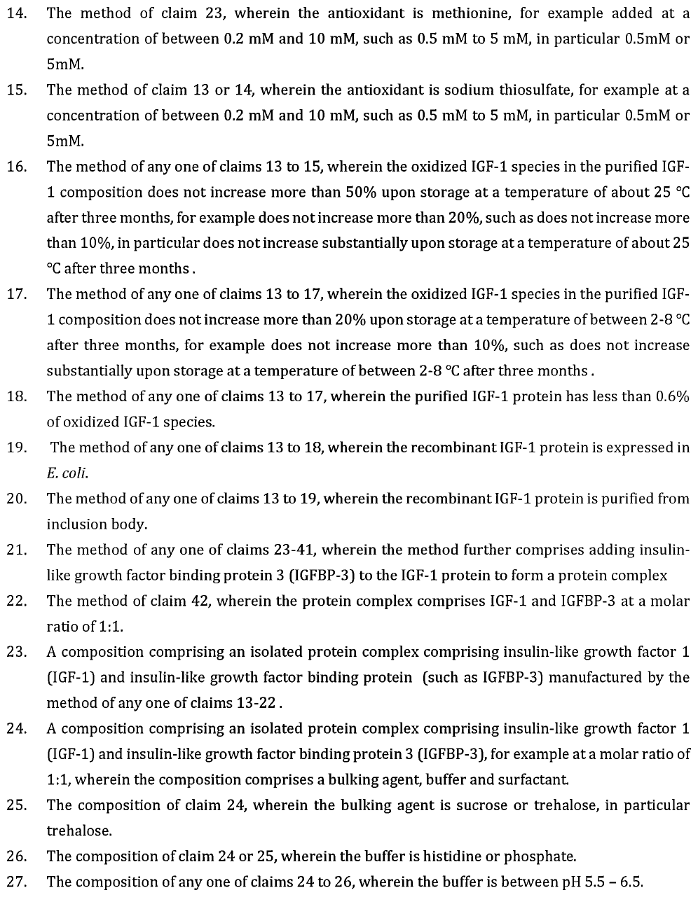

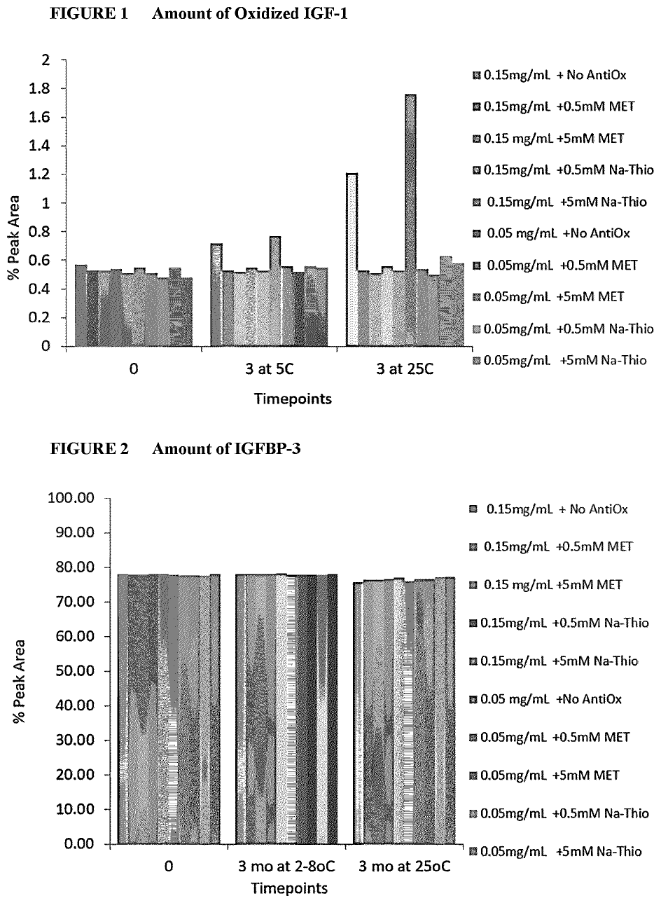

In this example, L-methionine and sodium thiosulfate are used as exemplary antioxidants at 5 mM and 50 mM concentrations. Mecasermin rinfabate (IGF-1/rhIGFBP-3) is used at protein concentrations of 0.15 mg/mL and 0.50 mg/mL. Stability was measured after 3 months of storage at 2-8° C. and 25° C. The percent oxidized species (ox rhIGF-1, FIG. 1), rhIGF-1 (FIG. 2) and rhIGFBP-3 (FIG. 3) were measured by RP-UPLC after 3 months at 2-8° C. (e.g. 5° C.) and 25° C.

TABLE 1
Measuring Ox rhIGF-1 (% Area) after 3 months by RP-UPLC

| RP-UPLC | Baseline | 3 mo @ 2-8° C. | 3 mo @ 25° C. | % increase 25° C. |
|---|---|---|---|---|
| 0.15 mg/mL + No AntiOx | 0.57 | 0.72 | 1.21 | 112.3% |
| 0.15 mg/mL + 0.5 mM MET | 0.53 | 0.53 | 0.53 | 0.0% |
| 0.15 mg/mL + 5 mM MET | 0.53 | 0.52 | 0.51 | −3.8% |
| 0.15 mg/mL + 0.5 mM Na-Thio | 0.54 | 0.55 | 0.56 | 3.7% |
| 0.15 mg/mL + 5 mM Na-Thio | 0.51 | 0.53 | 0.53 | −7.0% |
| 0.05 mg/mL + No AntiOx | 0.55 | 0.77 | 1.76 | 220.0% |
| 0.05 mg/mL + 0.5 mM MET | 0.51 | 0.56 | 0.54 | 5.9% |
| 0.05 mg/mL + 5 mM MET | 0.48 | 0.52 | 0.50 | 4.2% |
| 0.05 mg/mL + 0.5 mM Na-Thio | 0.55 | 0.56 | 0.63 | 10.5% |
| 0.05 mg/mL + 5 mM Na-Thio | 0.48 | 0.55 | 0.58 | 20.8% |

The results showed that the higher concentration (0.15 mg/mL) of protein was less susceptible to oxidative degradation, and that both methionine and sodium thiosulfate were effective at all concentrations at lowering increases in the oxidized rhIGF-1 species. At lower protein concentrations, the L-methionine showed higher efficacy as antioxidant.

TABLE 2
Measuring rhIGF-1 (% Area) after 3 months by RP-UPLC

| RP-UPLC | Baseline | 3 mo @ 2-8° C. | 3 mo @ 25° C. |
|---|---|---|---|
| 0.15 mg/mL + No antioxidant | 21.00 | 20.51 | 19.66 |
| 0.15 mg/mL + 0.5 mM MET | 21.27 | 20.82 | 20.52 |
| 0.15 mg/mL + 5 mM MET | 21.10 | 20.84 | 20.48 |
| 0.15 mg/mL + 0.5 mM Na-thio | 21.06 | 20.83 | 20.44 |
| 0.15 mg/mL + 5 mM Na-thio | 21.02 | 20.79 | 20.66 |
| 0.05 mg/mL + No antioxidant | 21.24 | 20.73 | 19.06 |
| 0.05 mg/mL + 0.5 mM MET | 21.41 | 20.94 | 20.63 |
| 0.05 mg/mL + 5 mM MET | 21.57 | 21.02 | 20.78 |
| 0.05 mg/mL + 0.5 mM Na-thio | 21.36 | 21.00 | 20.65 |
| 0.05 mg/mL + 5 mM Na-thio | 21.57 | 21.02 | 20.31 |

TABLE 3
Measuring rhIGF-BP3 (% Area) after 3 months by RP-UPLC

| RP-UPLC | Baseline | 3 mo @ 2-8° C. | 3 mo @ 25° C. |
|---|---|---|---|
| 0.15 mg/mL + No antioxidant | 77.94 | 78.07 | 75.87 |
| 0.15 mg/mL + 0.5 mM MET | 77.86 | 78.05 | 76.43 |
| 0.15 mg/mL + 5 mM MET | 77.93 | 77.98 | 76.4 |
| 0.15 mg/mL + 0.5 mM Na-thio | 78.00 | 78.01 | 76.68 |
| 0.15 mg/mL + 5 mM Na-thio | 78.10 | 78.16 | 76.97 |
| 0.05 mg/mL + No antioxidant | 77.90 | 77.85 | 76.04 |
| 0.05 mg/mL + 0.5 mM MET | 77.62 | 77.88 | 76.69 |
| 0.05 mg/mL + 5 mM MET | 77.69 | 77.8 | 76.61 |
| 0.05 mg/mL + 0.5 mM Na-thio | 77.60 | 77.9 | 77.15 |
| 0.05 mg/mL + 5 mM Na-thio | 77.94 | 77.96 | 77.26 |

### Example 2

Measuring Oxidized rIGF-1 after the addition of Antioxidants to an IGF-1/IGFBP-3 formulation by SEC-HPLC

This example illustrates the amount of oxidized species in a formulation comprising insulin like growth factor-1/insulin-like growth factor binding protein-3 (rhIGF-1/rhIGFBP-3) complex with and without the addition of antioxidants.

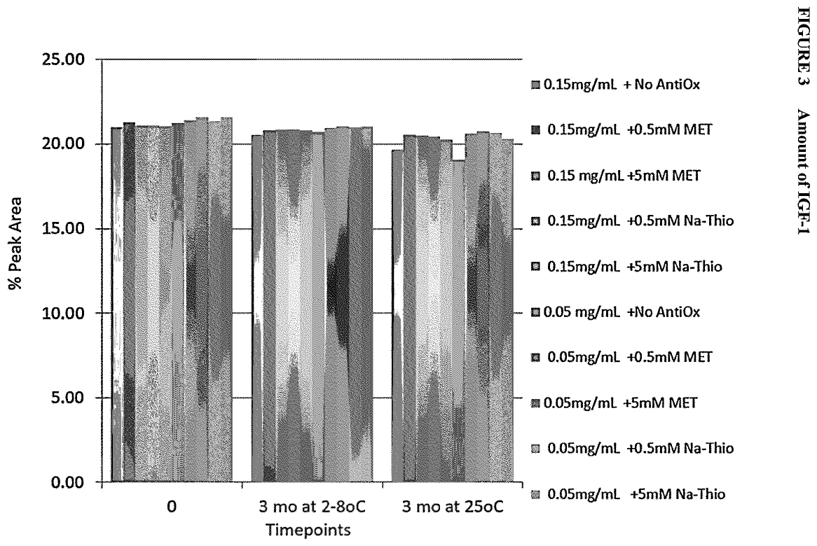

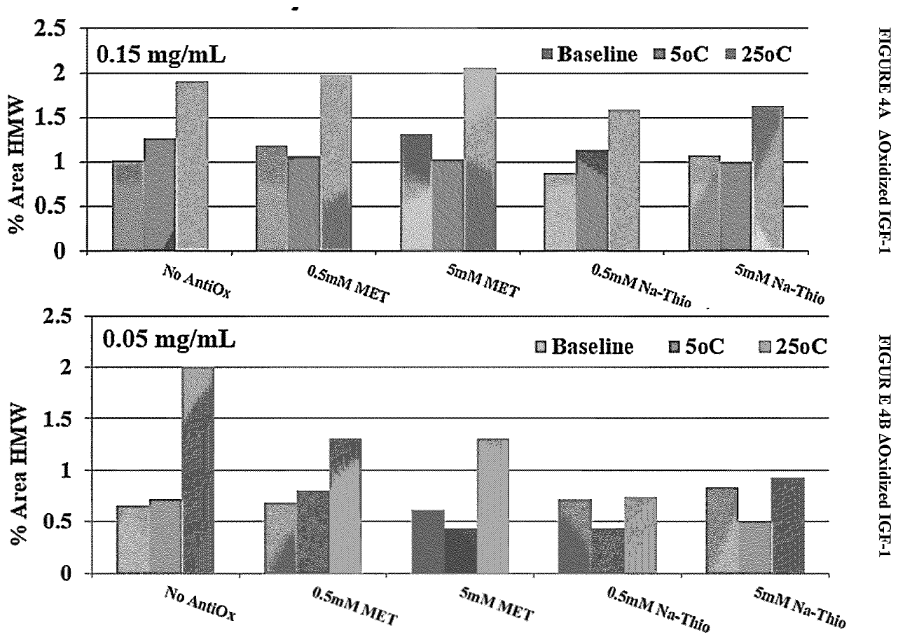

In this example, L-methionine and sodium thiosulfate are used as exemplary antioxidants at 5 mM and 50 mM concentrations. Mecasermin rinfabate (IGF-1/rhIGFBP-3) is used at protein concentrations of 0.15 mg/mL and 0.50 mg/mL. Stability was measured after 3 months of storage at 2-8° C. and 25° C. The percent oxidized species (ox rhIGF-1), rhIGF-1 and rhIGFBP-3 are measured by SEC-HPLC at baseline and after 3 months at 2-8° C. (e.g. 5° C.) and 25° C. The changes in oxidized rhIGF-1 at baseline and after 3 months by SEC assessment are shown at protein concentration of 0.15 mg/mL (FIG. 4A) and 0.05 mg/mL (FIG. 4B). The changes in rhIGF-1 at baseline and after 3 months by SEC assessment are shown at protein concentration of 0.15 mg/mL (FIG. 5A) and 0.05 mg/mL (FIG. 5B). The changes in oxidized rhIGFBP-3 at baseline and after 3 months by SEC assessment are shown at protein concentration of 0.15 mg/mL (FIG. 6A) and 0.05 mg/mL (FIG. 6B).

TABLE 4
Measuring Ox rhIGF-1 (% Area) after 3 months by SEC-HPLC

| SEC-HPLC | Baseline | 3 mo @2-8° C. | 3 mo @25° C. | % increase 25° C. |
|---|---|---|---|---|
| 0.15 mg/mL + No AntiOx | 1.02 | 1.26 | 1.91 | 87% |
| 0.15 mg/mL + 0.5 mM MET | 1.18 | 1.06 | 1.98 | 68% |
| 0.15 mg/mL + 5 mM MET | 1.31 | 1.03 | 2.06 | 57% |
| 0.15 mg/mL + 0.5 mM Na-Thio | 0.87 | 1.13 | 1.58 | 82% |
| 0.15 mg/mL + 5 mM Na-Thio | 1.08 | 0.99 | 1.63 | 51% |
| 0.05 mg/mL + No AntiOx | 0.66 | 0.72 | 2.01 | 205.0% |
| 0.05 mg/mL + 0.5 mM MET | 0.69 | 0.80 | 1.31 | 90% |
| 0.05 mg/mL + 5 mM MET | 0.61 | 0.43 | 1.31 | 115% |
| 0.05 mg/mL + 0.5 mM Na-Thio | 0.72 | 0.44 | 0.74 | 3% |
| 0.05 mg/mL + 5 mM Na-Thio | 0.87 | 0.51 | 0.93 | 7% |

The results showed that the formulation is less susceptible to degradation at the higher protein concentration of 0.15 mg/mL as compared to 0.05 mg/mL. The results also showed that both methionine and sodium thiosulfate were effective at all concentrations.

Further, by SEC analysis, the results showed that at lower protein concentrations, sodium thiosulfate is somewhat more effective as an antioxidant as compared to methionine.

TABLE 5
Measuring rhIGF-1 (% Area) after 3 months by SEC-HPLC

| SEC-HPLC | Baseline | 3 mo @ 2-8° C. | 3 mo @ 25° C. | % increase 25° C. |
|---|---|---|---|---|
| 0.15 mg/mL + No AntiOx | 0.27 | 0.42 | 0.45 | 67% |
| 0.15 mg/mL + 0.5 mM MET | 0.32 | 0.39 | 0.45 | 41% |
| 0.15 mg/mL + 5 mM MET | 0.27 | 0.36 | 0.49 | 81% |
| 0.15 mg/mL + 0.5 mM Na-Thio | 0.22 | 0.24 | 0.36 | 64% |
| 0.15 mg/mL + 5 mM Na-Thio | 0.23 | 0.22 | 0.25 | 9% |
| 0.05 mg/mL + No AntiOx | 0.22 | 0.43 | 0.37 | 68% |
| 0.05 mg/mL + 0.5 mM MET | 0.33 | 0.52 | 0.34 | 3% |
| 0.05 mg/mL + 5 mM MET | 0.27 | 0.45 | 0.28 | 4% |
| 0.05 mg/mL + 0.5 mM Na-Thio | 0.25 | 0.14 | 0.28 | 12% |
| 0.05 mg/mL + 5 mM Na-Thio | 0.23 | 0.08 | 0.22 | −4% |

TABLE 6
Measuring rhIGFBP-3 (% Area) after 3 months by SEC-HPLC

| SEC-HPLC | Baseline | 3 mo @ 2-8° C. | 3 mo @ 25° C. | % decrease 25° C. |
|---|---|---|---|---|
| 0.15 mg/mL + No AntiOx | 98.76 | 98.32 | 97.64 | 1% |
| 0.15 mg/mL + 0.5 mM MET | 98.51 | 98.55 | 97.57 | 1% |
| 0.15 mg/mL + 5 mM MET | 98.42 | 98.61 | 97.45 | 1% |
| 0.15 mg/mL + 0.5 mM Na-thio | 98.91 | 98.63 | 98.07 | 1% |
| 0.15 mg/mL + 5 mM Na-thio | 98.69 | 98.79 | 98.12 | 1% |
| 0.05 mg/mL + No AntiOx | 99.06 | 98.86 | 97.64 | 1% |
| 0.05 mg/mL + 0.5 mM MET | 98.98 | 98.68 | 97.57 | 1% |
| 0.05 mg/mL + 5 mM MET | 99.11 | 99.12 | 97.45 | 1% |
| 0.05 mg/mL + 5 mM Na-thio | 99.02 | 99.41 | 98.07 | 0% |
| 0.05 mg/mL + 0.5 mM Na-thio | 98.91 | 99.41 | 98.86 | 0% |

The data showed that both L-methionine and the sodium thiosulfate are capable of acting as antioxidants.

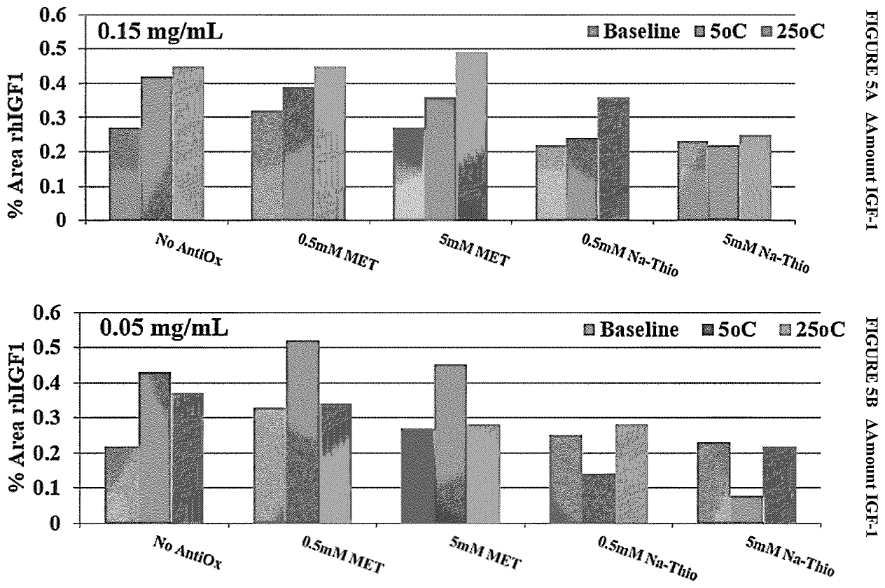

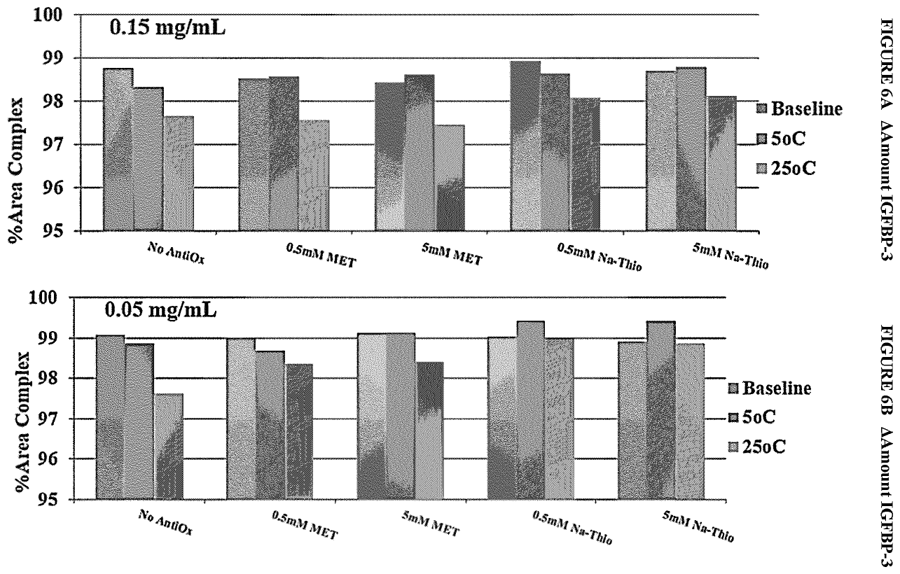

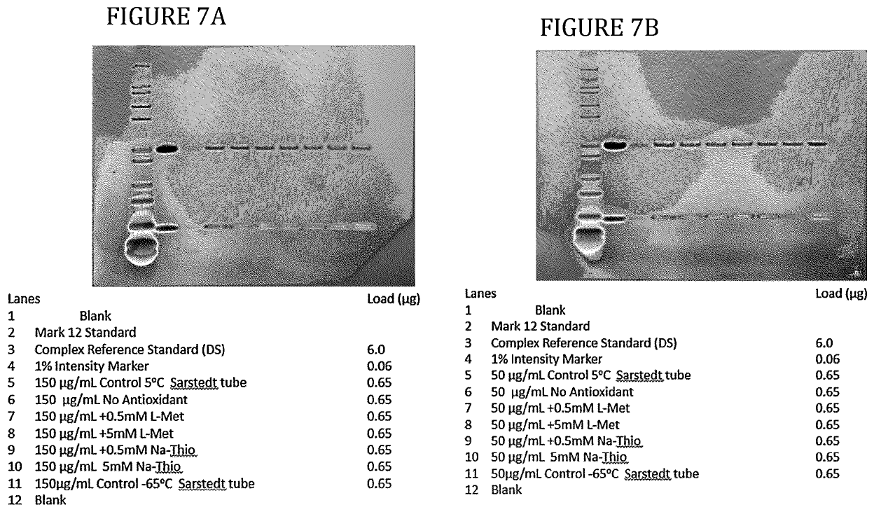

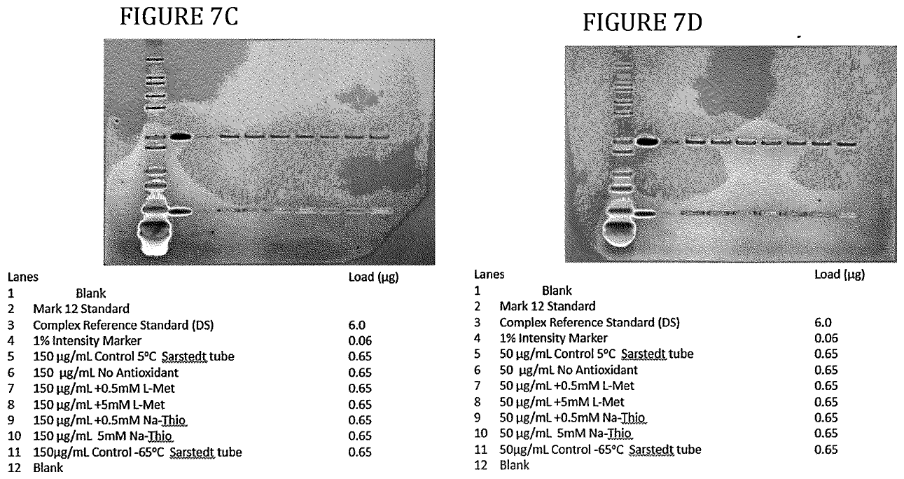

By SDS-PAGE, after 3 months at either 2-8° C. at 150 μg (FIG. 7A) and 50 μg protein concentrations (FIG. 7B) or 25° C. at 150 μg (FIG. 7C) and 50 μg (FIG. 7D), no significant protein degradation was observed.

### Example 3. Comparison of Oxidized rIGF-1 after the Addition of Antioxidants to an IGF-1/IGFBP-3 Formulation after Storage for 3 Months at Less than −65° C. and 2-8° C.

This example measures the amount of oxidized rIGF-1 after the addition of antioxidants to an IGF-1/IGFBP-3 formulation after storage for 3 months at less than −65° C. and 2-8° C.

TABLE 7
Measuring Ox rhIGF-1 (% Area) after 3 months by RP-UPLC

| | | | 3 mo at less |
|---|---|---|---|
| RP-UPLC | Baseline | 3 mo @ 2-8° C. | than −65° C. |
| 0.15 mg/mL + No AntiOx | 0.57 | 0.72 | NT |
| 0.15 mg/mL + No AntiOx | 0.57 | 0.60 | 0.54 |
| (1 mL/2 mL tube) | | | |
| 0.05 mg/mL + No AntiOx | 0.54 | 0.56 | NT |
| 0.05 mg/mL + No AntiOx | 0.54 | 0.64 | 0.56 |
| (1 mL/2 mL tube) | | | |

The data in this example showed that storing at less than −65° C. (freezing) is preferable to storing at 2-8° C.

TABLE 8
Measuring Ox rhIGF-1 (% Area) after 3 months by SEC-UPLC

| | | | 3 mo at less |
|---|---|---|---|
| SEC-UPLC | Baseline | 3 mo at 2-8° C. | than −65° C. |
| 0.15 mg/mL + No AntiOx | 1.02 | 1.26 | NT |
| 0.15 mg/mL + No AntiOx | 1.02 | 1.41 | 1.29 |
| (1 mL/2 mL tube) | | | |
| 0.05 mg/mL + No AntiOx | 0.66 | 0.72 | NT |
| 0.05 mg/mL + No AntiOx | 0.66 | 0.99 | 0.88 |
| (1 mL/2 mL tube) | | | |

### Example 4. Lyophilization of Mecasermin Rinfabate (IGF-1/IGFBP-3)

This example demonstrates the effect of lyophilization on percent oxidation and stability of IGF-1/IGFBP-3.

In this example, mecasermin rinfabate drug product formulations using various buffer excipients and bulking agents were lyophilized and physical properties and degradation was assessed.

In this example, the protein concentration of the IGF-1/IGFBP-3 complex was 0.15 mg/mL (150 μg/ml) and polysorbate was added at 0.015%. Three formulation buffers such as histidine chloride, sodium phosphate and sodium citrate buffers and two bulking agents, sucrose and trehalose, were compared (Table 9).

The results demonstrated that histidine buffer resulted in the smallest amount of high molecular weight (HMW) species formation.

TABLE 9
Buffers and Bulking agents in Mecasermin Rinfabate

| | Buffer | Bulking Agent |
|---|---|---|
| 1. | 10 mM citrate, pH 6.0 | 6% sucrose |
| 2. | 10 mM citrate, pH 6.0 | 6% trehalose |
| 3. | 10 mM histidine, pH 6.0 | 6% sucrose |
| 4. | 10 mM histidine, pH 6.0 | 6% trehalose |
| 5. | 10 mM phosphate, pH 6.0 | 6% sucrose |
| 6. | 10 mM phosphate, pH 6.0 | 6% trehalose |

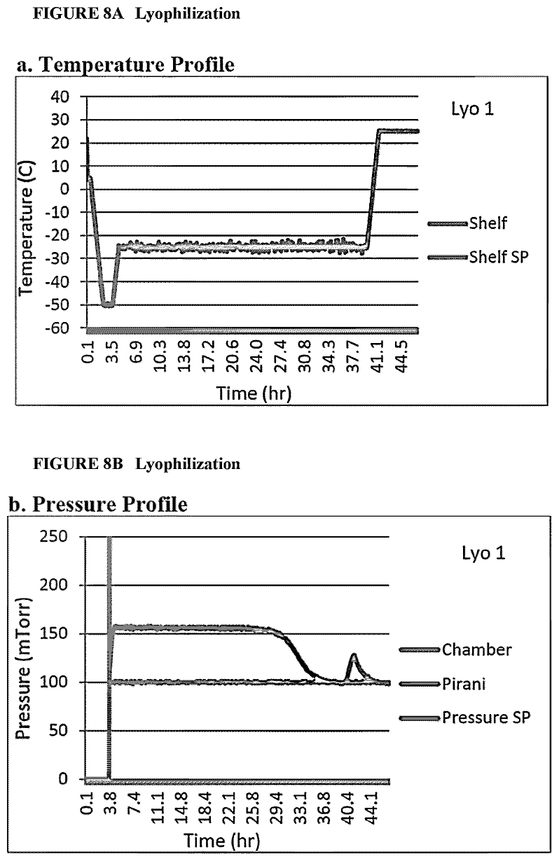

Lyophilization was carried out under a lyophilization cycle consisting of a primary drying temperature of −25° C. and a secondary drying temperature of 25° C. (Table 10). The temperature profile during the run is shown in FIG. 8A, and the pressure profile in FIG. 8B.

TABLE 10
Buffers and Bulking agents in Mecasermin Rinfabate

| | Buffer | Temperature | | Ramp Rate | Pressure | Time | |
|---|---|---|---|---|---|---|---|
| 1. | Cooling | 5° | C. | 1.0° C./min. | Atmospheric | 0.5 | hr |
| 2. | Freezing | −50° | C. | 0.5° C./min. | Atmospheric | 1 | hr |
| 3. | Primary | −25° | C. | 0.5° C./min. | 100 mTorr | 35 | hr |
| | drying | | | | | | |
| 4. | Secondary | 25° | C. | 0.5° C./min. | 100 mTorr | 6 | hr |
| | drying | | | | | | |

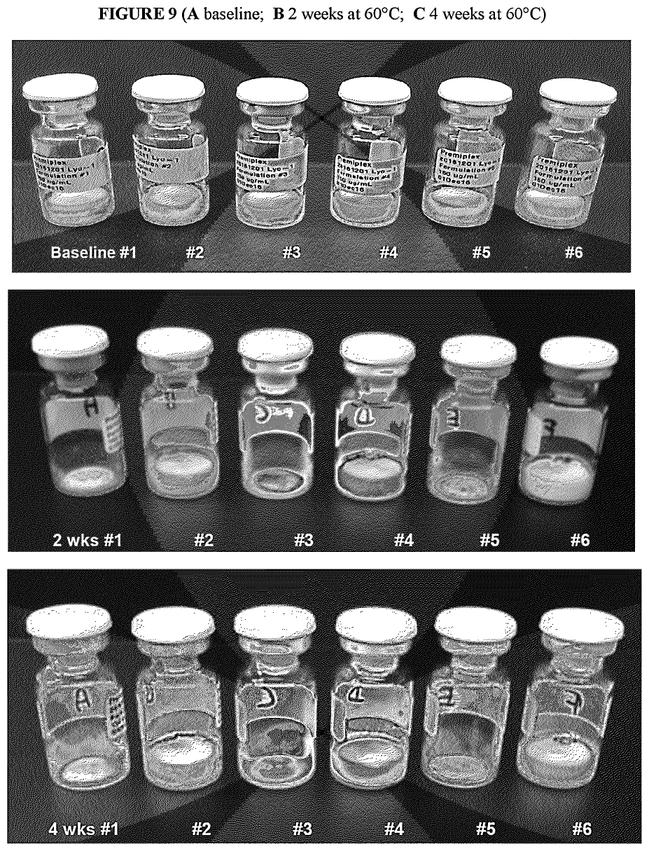

The lyophilization run resulted in homogeneous white cakes for all vials (Table 11, FIG. 9A, FIG. 9B and FIG. 9C). The lyophilized vials were reconstituted with 6.2 mL of normal saline and product quality was assessed by protein concentration with SoloVPE, pH, SE-UPLC, RP-UPLC and reduced SDS-PAGE. Cake appearance and reconstitution time were also assessed for the lyophilized formulations in addition to the osmolality that was assessed at baseline for the formulated liquid drug product.

TABLE 11
Cake appearance after 2 weeks of storage at 60° C.

| No. | Baseline | 2 week, 60° C. | 4 week, 60° C. |
|---|---|---|---|
| 1 | Firm, white, | Melted | Melted |
| | 40% shrinkage, plugged | | |
| 2 | Firm, white, | Unchanged | Unchanged |
| | 20% shrinkage, loose | | |
| 3 | Firm, white, | Melted | Melted |
| | 10% shrinkage, plugged | | |
| 4 | Firm, white, | Cracked cake | Unchanged |
| | 10% shrinkage, loose | | |
| 5 | Firm, white, | Melted | Melted |
| | 50% shrinkage, plugged | | |
| 6 | Firm, white, | Unchanged | Unchanged |
| | 10% shrinkage, plugged | | |

As seen from the results in Table 11, after 2 weeks of storage at 60° C., the cakes containing sucrose melted, while the trehalose containing cakes appeared unchanged.

TABLE 12
Reconstitution Time and Osmolality

Baseline

| | | | |
|---|---|---|---|
| | Osmolality | 2 wk, | 4 wk, |
| Sample | (before | 60° C. | 60° C. |

| | | |
|---|---|---|
| No. | lyophilization) | Reconstitution time |

| | | | | | | |
|---|---|---|---|---|---|---|
| 1 | 212 | <15 sec | 60 | sec | 90 | sec |
| 2 | 198 | <15 sec | <20 | sec | <20 | sec |
| 3 | 199 | <15 sec | 40 | sec | 30 | sec |
| 4 | 183 | <15 sec | <20 | sec | <20 | sec |
| 5 | 205 | <15 sec | 60 | sec | 60 | sec |
| 6 | 190 | <15 sec | <20 | sec | <20 | sec |

The sucrose containing cakes needed increased reconstitution time, while the reconstitution time was practically unchanged for trehalose containing cakes (Table 12).

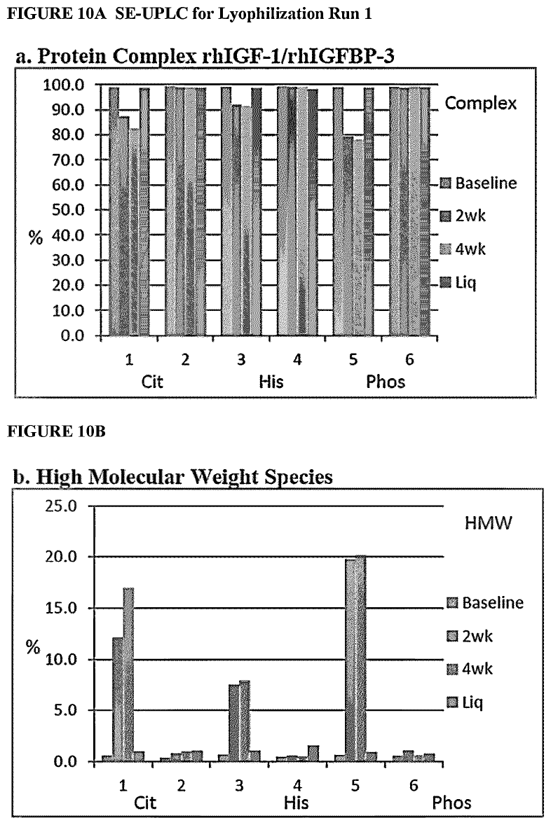

SE-UPLC separates the main peak from the high molecular weight aggregate peak (FIG. 10A). Analysis by SE-UPLC showed that the use of trehalose as a bulking agent resulted in better protein stability than the use of sucrose. The HMW peak percentages for the sucrose formulations after 4 weeks of storage at 60° C. ranged from 7.9% for formulation #3 to 20.2% for formulation #5 (FIG. 10B). The increase in aggregation indicates that trehalose is a better cryoprotectant than sucrose or as a result of the sucrose containing lyophilized cakes collapsing due to, for example, the remaining moisture in the cake. A comparison of the results for sodium citrate, histidine chloride, and sodium phosphate buffers showed that the drug product formulated in histidine buffer resulted in the smallest amount of HMW species formation.

Lyophilization of mecasermin rinfabate formulations using either sucrose or trehalose as bulking agents resulted in white, uniform cakes. Some cake shrinkage was observed (a ˜10%-20% visual shrinkage for trehalose containing formulations and ˜10-50% for sucrose containing formulations). Thermal stress storage for 4 weeks at 60° C. was used to assess degradation of lyophilized drug product. Degradation was least in formulations comprising trehalose.

The data from this example showed that trehalose performed better than sucrose as a bulking agent for a lyophilized presentation and that histidine and phosphate buffers are preferable buffers when compared to the citrate buffer. The data also showed that the formulation in the liquid form before lyophilization is stable for 48 hours in refrigerated condition.

### Example 5. Lyophilization of Mecasermin Rinfabate (IGF-1/IGFBP-3)

This example demonstrates the effect of pH on lyophilization of IGF-1/IGFBP-3 in histidine buffer.

Mecasermin rinfabate (IGF-1/IGFBP-3) at three pH values of 5.5, 6.0, and 6.5 in histidine buffer were compared (Table 13). The protein concentration prior to lyophilization was 0.15 mg/mL and polysorbate was added at 0.015%. Trehalose was used as a bulking agent. The concentration of trehalose was reduced to 4% to reduce overall osmolality. Formulation #4 in Table 5 contained 1 mM methionine to assess the impact of methionine on oxidation levels.

TABLE 13
Drug Product Formulations (Protein 150 μg/mL all samples)

| | Buffer | | | |
|---|---|---|---|---|
| Sample | 10 mM histidine | Surfactant | Bulking agent | Methionine |
| 1. | pH 5.5 | 0.015% P20 | 4% trehalose | — |
| 2. | pH 6.0 | 0.015% P20 | 4% trehalose | — |
| 3. | pH 6.5 | 0.015% P20 | 4% trehalose | — |
| 4. | pH 6.0 | 0.015% P20 | 4% trehalose | 1 mM |
| 5. | Ph 6.0 | 0.015% P20 | 4% trehalose | 1 mM |

Lyophilization run was carried out under a lyophilization cycle consisting of a primary drying temperature of −20° C. and a secondary drying temperature of 25° C. using a lyostar lyophilizer (Table 14).

TABLE 14
Buffers and Bulking agents in Mecasermin Rinfabate

| | Buffer | Temperature | | Ramp Rate | Pressure | Time | |
|---|---|---|---|---|---|---|---|
| 1. | Cooling | 5° | C. | 1.0° C./min. | Atmospheric | 0.5 | hr |
| 2. | Freezing | −50° | C. | 0.5° C./min. | Atmospheric | 1 | hr |
| 3. | Primary | −25° | C. | 0.5° C./min. | 100 mTorr | 35 | hr |
| | drying | | | | | | |
| 4. | Secondary | 25° | C. | 0.5° C./min. | 100 mTorr | 6 | hr |
| | drying | | | | | | |

The lyophilized drug product vials were placed at the thermal stress condition of 40° C. for 2 weeks and 4 weeks and 60° C. for 2 weeks. One vial of each formulation was held for 48 hours at room temperature as the liquid control without lyophilization. The lyophilization run resulted in homogeneous firm white cakes and showed shrinkage by visual assessment (20% at baseline, 20-30% after thermal stress storage). The lyophilized vials were reconstituted with 6.2 mL of normal saline. The reconstitution time was below 20 sec at baseline for all formulations.

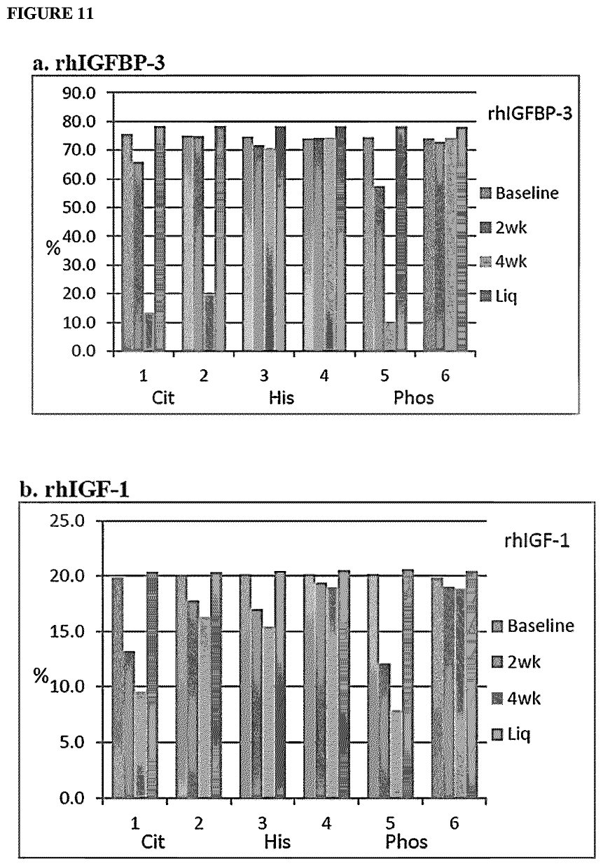

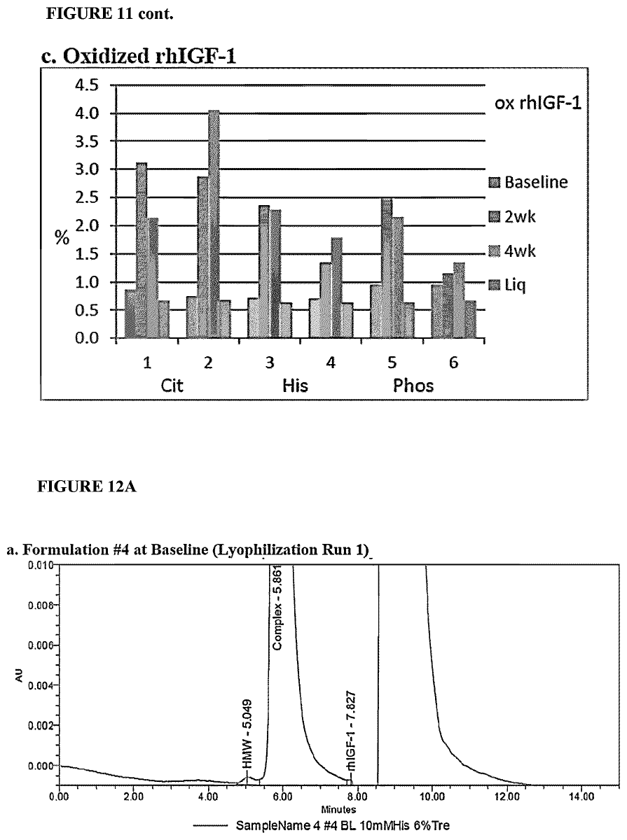

SE-UPLC analysis showed that the % peak areas of complex of rhIGF-1 and rhIGFBP-3 were the highest at pH values 6.0 and 6.5 and the high molecular weight species was the lowest after 4 weeks of thermal storage at pH 6.0, followed by pH 6.5 (FIG. 11A). The addition of methionine showed low amount of high molecular weight species for the liquid sample that was held for 48 hours at room temperature (FIG. 11A).

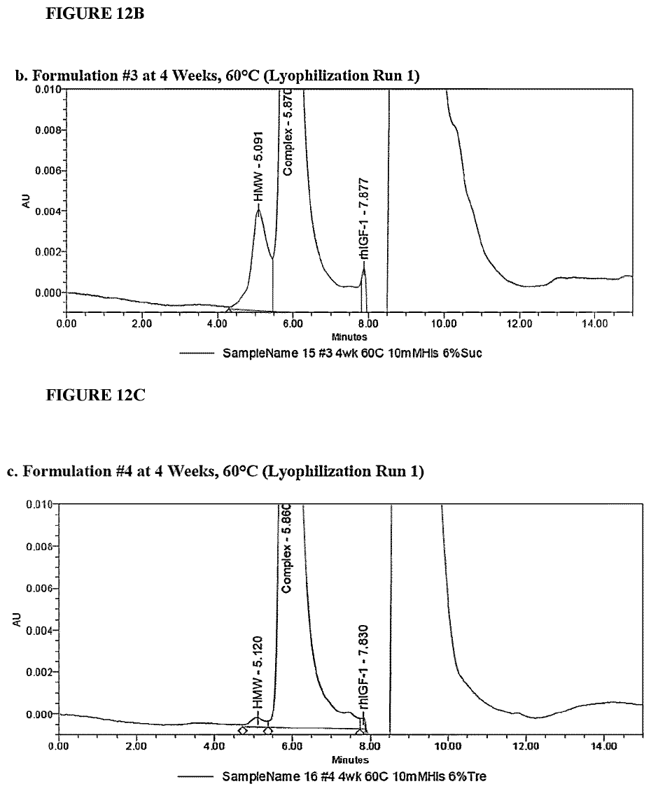

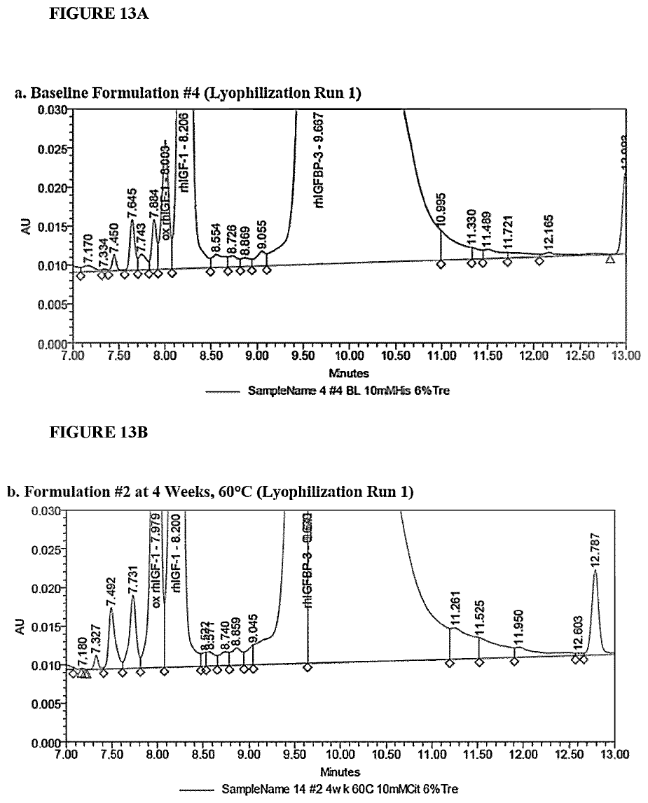

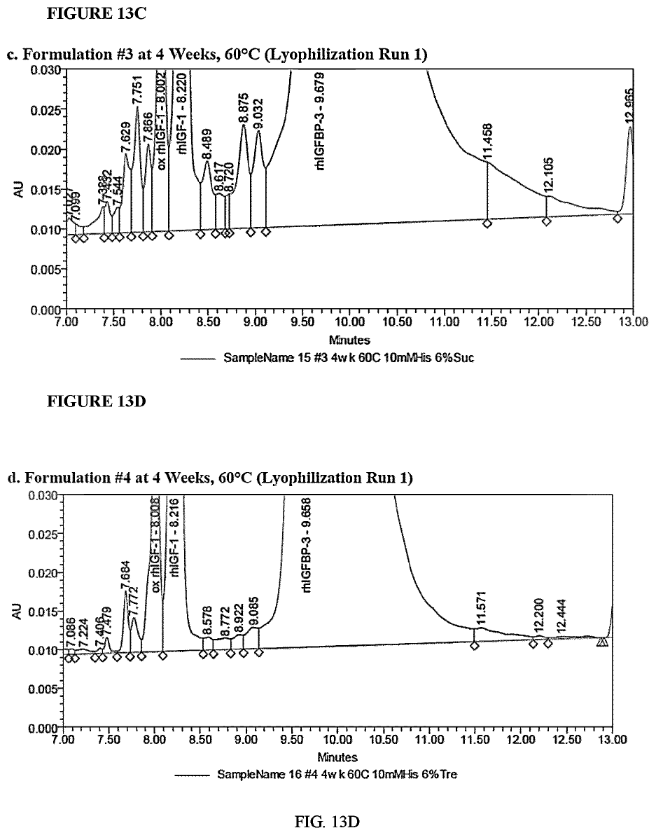

A representative lyophilization run at baseline is shown at FIG. 12A. Two representative formulations after 4 weeks of storage at 60° C. (FIG. 12B and FIG. 12C). A representative lyophilization run at baseline at FIG. 13A, FIG. 13B, FIG. 13C show representative formulations after 4 weeks of storage at 60° C.

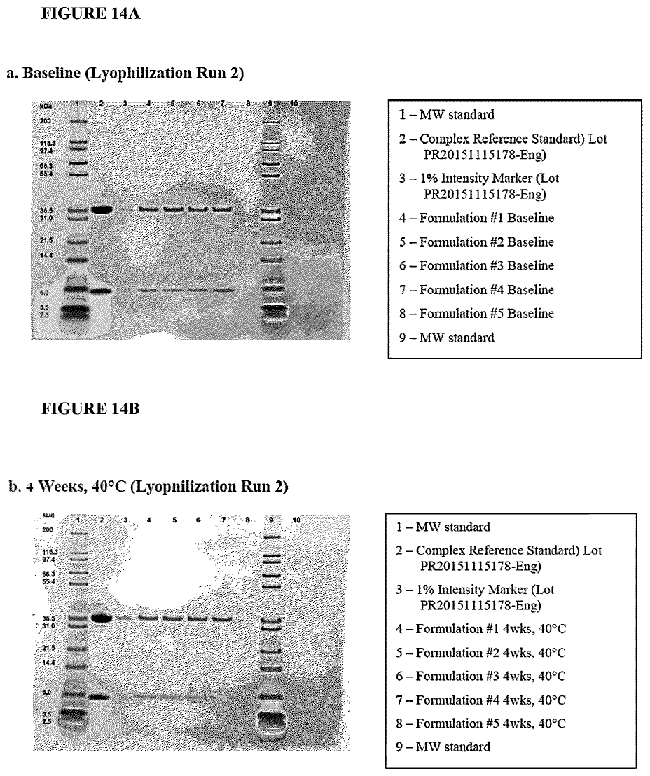

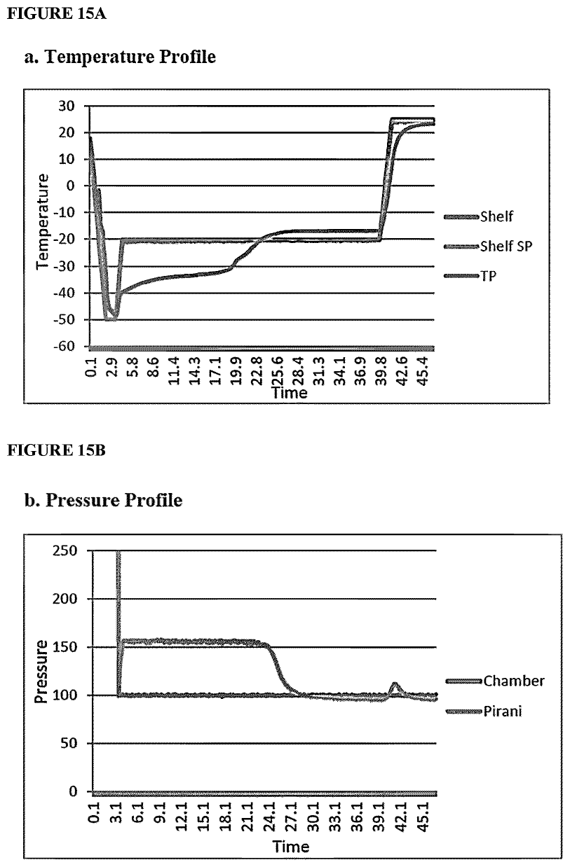

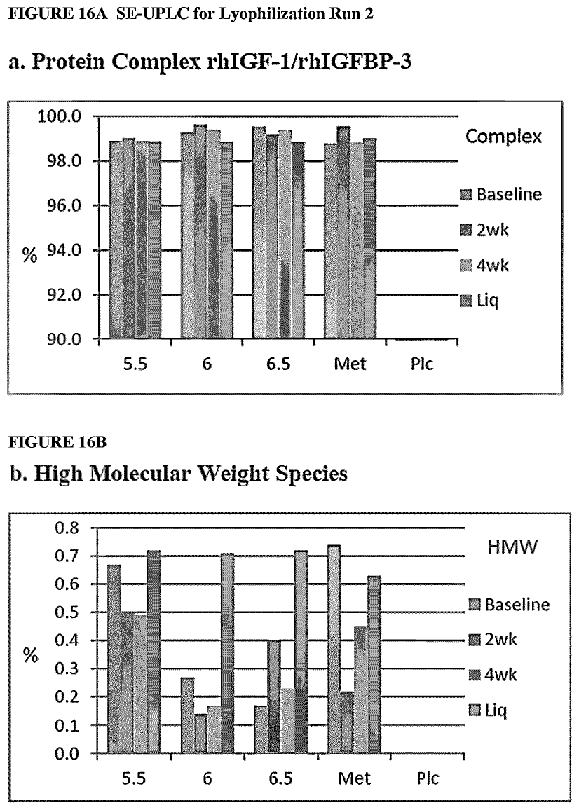

An SDS PAGE gel of representative lyophilized formulations at baseline is shown at FIG. 14A, in comparison to an SDS PAGE gel of representative lyophilized formulations after 4 weeks of storage at 40° C. (FIG. 14B). FIG. 15A shows temperature profile of an exemplary lyophilization run. FIG. 15B shows a pressure profile of an exemplary lyophilization run. FIG. 16A shows a graph of the amount of percent rhIGF-1/rhIGFBP-3 protein complex from an exemplary lyophilization run. FIG. 16B shows a graph of the amount of high molecular weight species from an exemplary lyophilization run.

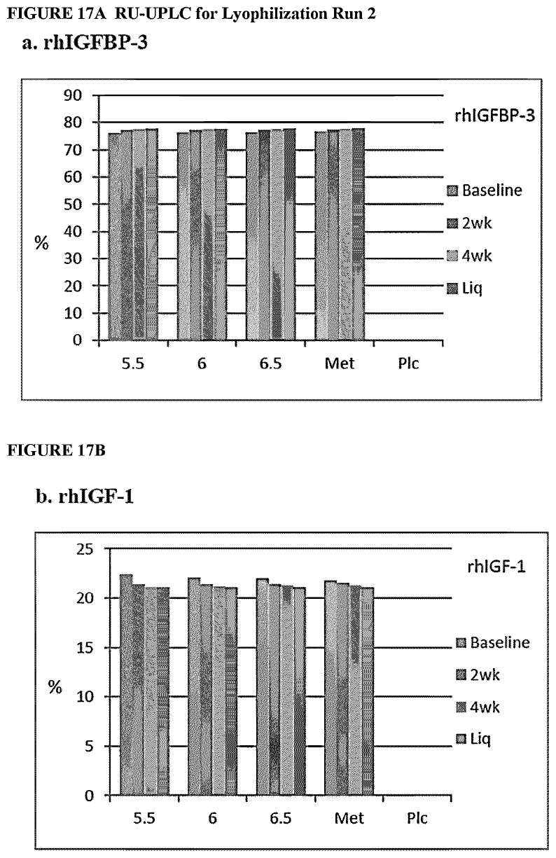

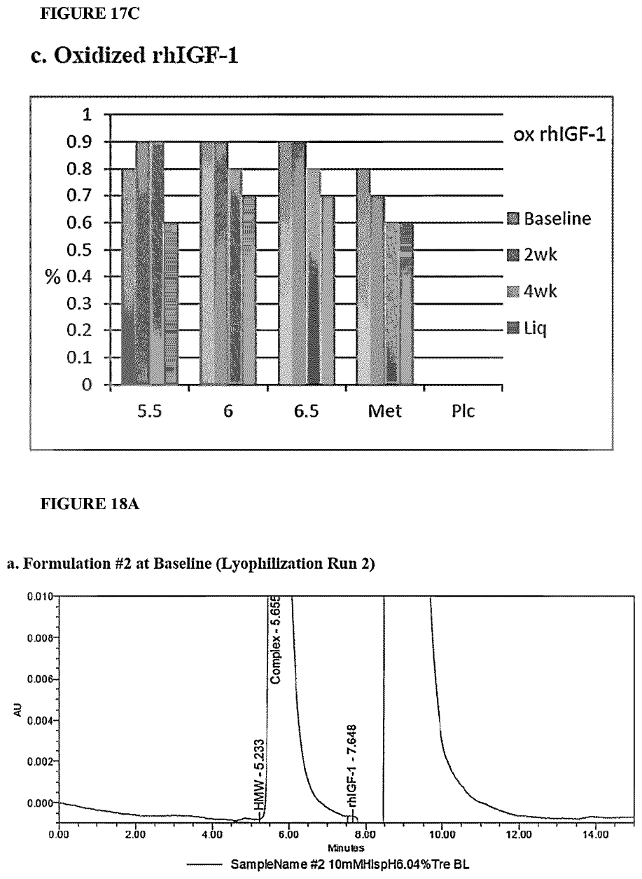

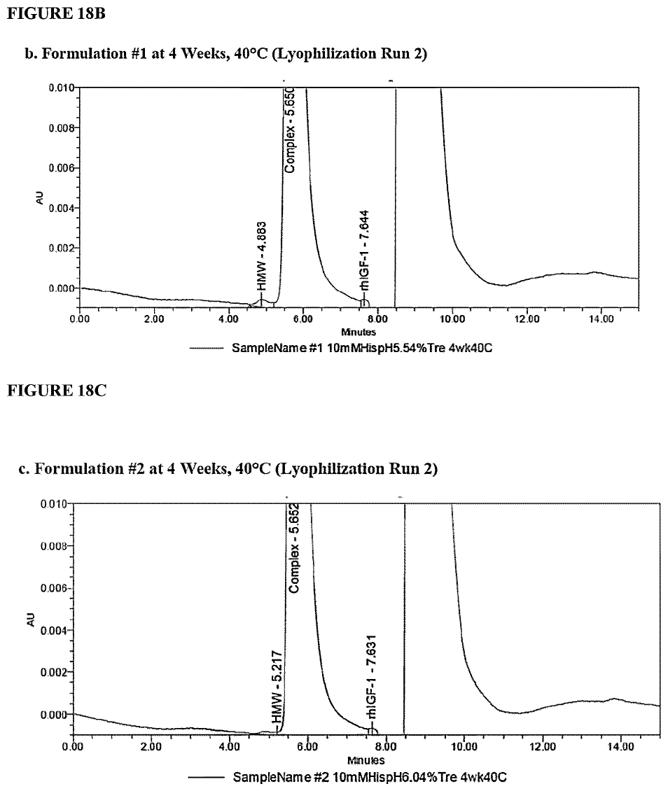

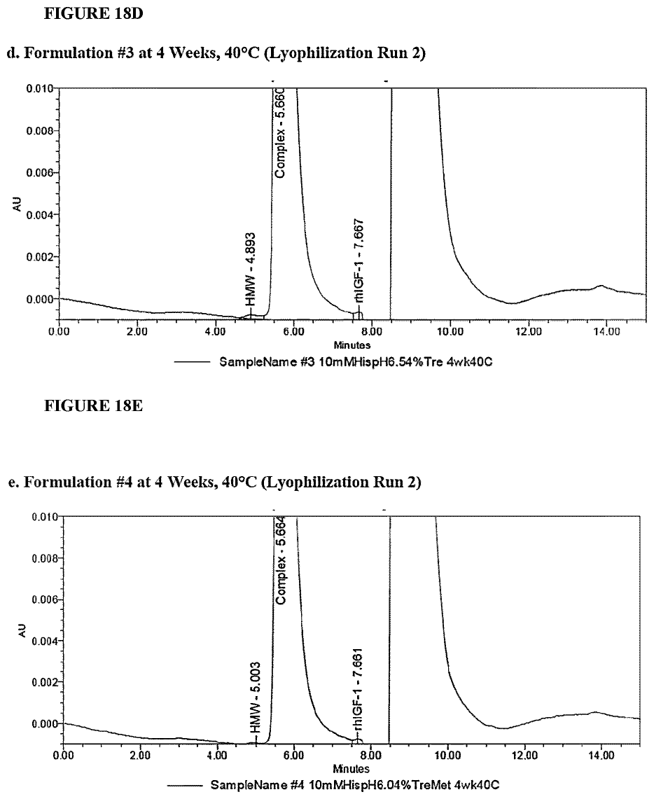

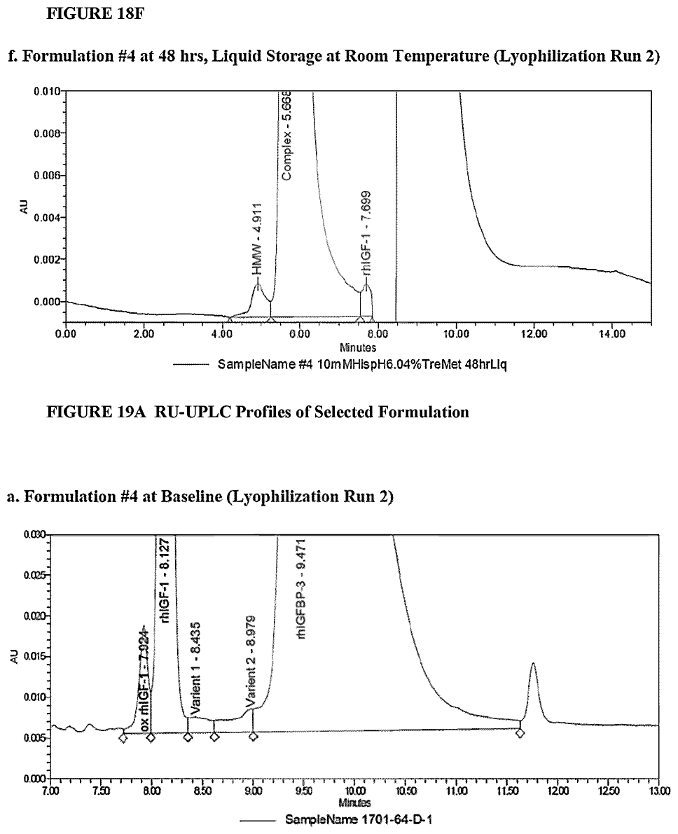

RP-UPLC data of all four formulations containing protein were similar (FIG. 17A). A reduction of oxidation was observed with the sample containing 1 mM methionine (FIG. 17A). FIG. 17B shows a graph of the amount of rhIGF-1 as measured by RP-UPLC at baseline, after 2 weeks of storage, after 4 weeks of storage, compared to liquid formulation. FIG. 17C shows a graph of the amount of oxidized rhIGF-1 as measured by RP-UPLC at baseline, after 2 weeks of storage, after 4 weeks of storage, compared to liquid formulation. No significant difference was observed between pH 5.5, 6.0 and 6.5. FIG. 18A shows a graph of a representative lyophilized formulation at baseline. Graphs of representative formulation after 4 weeks of storage at 40° C. are shown in FIG. 18B, FIG. 18C, FIG. 18D and FIG. 18E. FIG. 18F shows a graph of a representative formulation after 48 hours of liquid storage at room temperature.

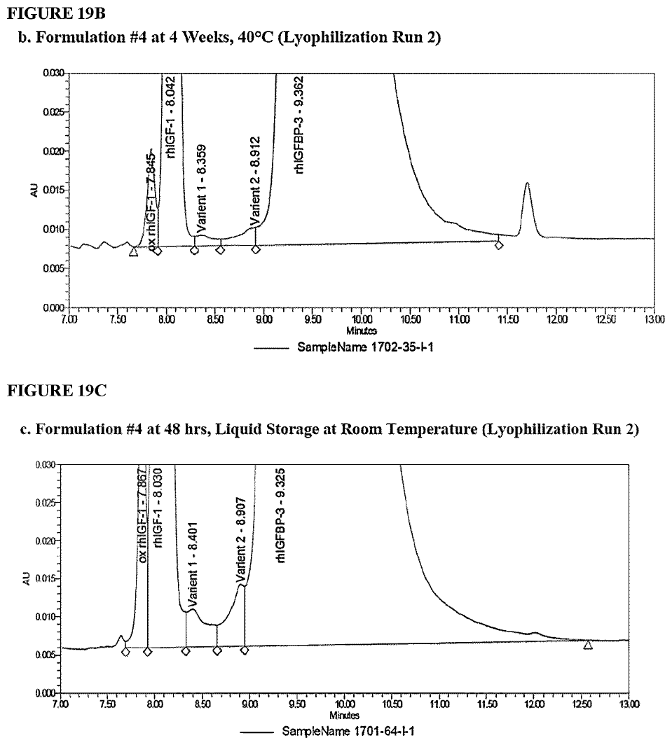

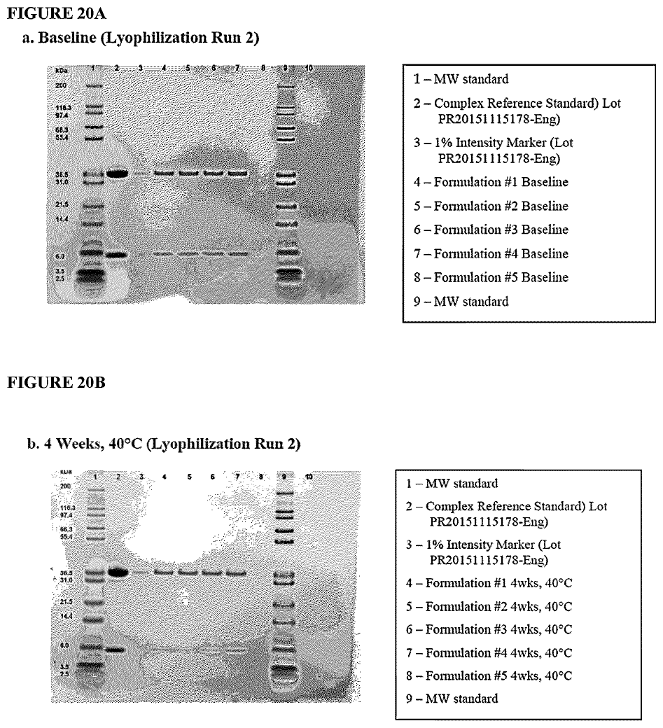

A graph of a representative formulation at baseline is shown in FIG. 19A in comparison to a graph of a representative formulation after 4 weeks of storage at 40° C. (FIG. 19B). FIG. 19C shows a graph of a representative formulation after 48 hours of liquid storage at room temperature. Analysis by SDS-PAGE (Coomassie) did not show any degradation (FIG. 20A and FIG. 20B). The measured pH values were in the range of ±0.1 units from the expected value after stress storage (Table 12).

Overall, the data in this example showed that the pH range from 5.5 to 6.5 is suitable for the formulation and that the addition of a small amount of methionine (1 mM) lowered the propensity for oxidation for the lyophilized presentation and the liquid control.

While certain compounds, compositions and methods described herein have been described with specificity in accordance with certain embodiments, the following examples serve only to illustrate the compounds of the invention and are not intended to limit the same.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications, websites and other reference materials referenced herein to describe the background of the invention and to provide additional detail regarding its practice are hereby incorporated by reference.

1-27. (canceled)

28. A method of manufacturing an insulin-like growth factor 1 (IGF-1) protein complex comprising steps of: providing a composition comprising recombinant IGF-1 expressed from E. coli; adding an antioxidant to the composition; and
purifying from the recombinant IGF-1 protein from an inclusion body,
wherein the antioxidant is nitrogen.

29. A method according to claim 28, wherein oxidation is reduced by repeated pull vacuum.

30. A method according to claim 28, wherein the method further comprises adding insulin-like growth factor binding protein 3 (IGFBP-3) to the IGF-1 protein to form a protein complex.

31. A method according to claim 30, wherein the protein complex comprises IGF-1 and IGFBP-3 at a molar ratio of 1:1.

32. A method of manufacturing according to claim 28, wherein reduced accumulation of oxidized species and improved stability is achieved through the use of a contact surface other than stainless steel.

33. A method according to claim 30, wherein a single-use bag is employed during compounding the complex with a formulation solution.

34. A method of manufacturing according to claim 28, wherein the composition is a liquid formulation.

35. A method according to claim 34, wherein the formulation is isotonic.

36. A method according to claim 35, wherein the formulation does not comprise any ingredients that are toxic to a premature infant.

37. A method according to claim 36, wherein the formulation does NOT comprise a preservative.

38. A method according to claim 28, wherein the composition comprises a surfactant selected from polysorbate 20 and polysorbate 80.

39. A method according to claim 38, wherein the polysorbate 20 is present at 0.005%.

40. A method according to claim 38, wherein the polysorbate 80 is present at 0.005%.

41. A method according to claim 28, wherein the composition is at a pH between about 5.0 and 6.0

42. A method according to claim 33, wherein the formulation comprises:
a. rIGF-1/rIGFBP-3 is in equimolar amounts,
b. polysorbate 20 surfactant at a concentration of about 0.005%,
c. and a buffer comprising sodium acetate, acetic acid, and/or sodium chloride,
wherein
the composition has a pH of about 5.3-5.8,
the rIGF-1/IGFBP-3 is at a concentration of about 50 micrograms/mL, and
less than 0.6% of the IGF-1 exists as oxidized species.

43. A method according to claim 42, wherein the liquid formulation is contained in a vial.

44. A method according to claim 43, wherein the vial is glass.

45. A method according to claim 44, wherein the vial is under nitrogen.

46. A method according to claim 44, further comprising the step of providing an article of manufacture comprising packaging material of a pharmaceutical agent and said formulation.
